(19) United States
(12) Patent Application Publication
(10) Pub. No.: US 2023/0225984 A1
(43) Pub. Date: Jul. 20, 2023

(54) MONO- AND BIS-NITROSYLATED ALKYL POLYOLS FOR THERAPEUTIC USE

(71) Applicant: ATTGENO AB, Solna (SE)

(72) Inventors: Per Håkan Agvald, Stockholm (SE); Kristofer Bo Ingemar Nilsson, Örebro (SE); Leif Christofer Adding, Åkersberga (SE)

(21) Appl. No.: 17/927,780

(22) PCT Filed: May 27, 2021
(86) PCT No.: PCT/EP2021/064262

§ 371 Date: Nov. 25, 2022

(30) Foreign Application Priority Data
May 27, 2020 (GB) 2007928.1

(51) Int. Cl. A61K 31/045; C07C 215/06; A61P 31/12; A61P 31/04; A61P 33/00

(52) U.S. Cl. CPC A61K 31/045; C07C 215/06; A61P 31/12; A61P 31/04; A61P 33/00

(57) ABSTRACT

The present invention relates to the use of mono- and bis nitrosylated propanediols, and compositions and formulations comprising the same, in methods for the treatment of a microbial infection.

### FIELD OF THE INVENTION

The present invention relates to the use of mono- and bis-nitrosylated alkyl polyols, and compositions and formulations comprising the same, in methods for the treatment of a microbial infection.

### BACKGROUND

The listing or discussion of an apparently prior-published document in this specification should not necessarily be taken as an acknowledgment that the document is part of the state of the art or common general knowledge.

It is estimated that diseases caused by microbial infections kill over 17 million people a year. The most common deadly infections include pneumonia, diarrhoeal diseases, tuberculosis, malaria, hepatitis, HIV/AIDS, measles, neonatal tetanus, pertussis and intestinal worms (WHO, The World Health Report 1996—Fighting disease, fostering development). Typically, such diseases are caused by infection with parasitic, bacteria, fungi or viral particles.

A bacteria is a microscopic single-celled organism which belongs to the domain of prokaryotes. Bacteria are characterised by the lack of a nucleus as their DNA floats freely inside the organism, which is surrounded by a lipid membrane and a cell wall consisting of peptidoglycan. Although certain bacteria, such as the mycoplasmas, do not have a cell wall at all and conversely some have a third, outermost protective layer called the capsule. They have a number of shapes including spheres, rods and spirals and some species contain flagella which helps them move. It is estimated that there are 10⁷ to 10⁹ different bacteria species on earth. Animals live in a symbiotic relationship with bacteria and many functions like the human digestive system would not work without microbes including bacteria that live in the gut and bowel. However, there are around 100 bacteria species which are pathogenic to humans and cause severe illness and even death. Bacteria often infect humans through food or water but human or animal to human is also possible.

A fungus is a microscopic eukaryotic organism with the characteristic that it contains chitin in its cell walls. Unlike bacteria, fungi contain membrane-bound organelles and a clearly defined nucleus. Fungi live either in single-celled organisms, i.e. yeast, or multicellular organisms, i.e. mould or mushrooms. Humans have found fungi useful in a number of applications like food and medicine but like bacteria, there are several fungi that cause severe illness and death in humans. Fungal diseases usually spread through direct contact or inhalation. A large number of fungal infections develop in the upper layers of the skin, and some progress to the deeper layers. Systemic fungal infections, such as pneumonia and other infections in the body, may also occur by inhalation of yeast or mould spores.

A parasite is an organism that lives on or in other organisms (host organisms) causing it harm. Parasites that infect humans mainly include protozoans, helminths and ectoparasites. Protozoans are single-celled eukaryotes, including single celled fungi, that feed on organic matter. Helminths are large macro parasites and can usually be seen with a human eye. Examples of helminths are intestinal worms that live in the gastrointestinal tract and schistosomes that live in the blood vessels of its host. Ectoparasites include parasites that live mainly on the surface of their host organism. Parasites usually infect humans through direct contact, contaminated food and water or through an intermediate host (e.g. a mosquito).

Nitric oxide has been shown to be produced by a number of different cell types in response to cytokine stimulation and thus has been found to play a role in immunologically mediated protection against a growing list of protozoan and helminth parasites in vitro and in animal models. The biochemical basis of its effects on the parasite targets appears to involve primarily inactivation of enzymes crucial to energy metabolism and growth, although it has other biologic activities as well. NO is produced not only by macrophages and macrophage-like cells commonly associated with the effector arm of cell-mediated immune reactivity but also by cells commonly considered to lie outside the immunologic network, such as hepatocytes and endothelial cells, which are intimately involved in the life cycle of a number of parasites. NO production is stimulated by gamma interferon in combination with tumor necrosis factor alpha or other secondary activation signals and is regulated by a number of cytokines (especially interleukin-4, interleukin-10, and transforming growth factor beta) and other mediators, as well as through its own inherent inhibitory activity (James, Microbiology rev. 1995 December; 59(4): 533-547).

A virus is a small organism comprising genetic material (DNA or RNA) that is capable of infecting a biological organism. A virus invades and attaches itself to a living cell, after which it multiplies to produce more virus particles (virions), which attach to and enter susceptible cells. The virus may either kill a cell or alter its functions leading to the infection of other cells. This will then generally lead to what is termed as viral diseases (or a viral infection). In general, viruses can be transmitted in various ways, including contact with infected individuals or their bodily secretions, animals such as arthropods, or inanimate objects. Viruses can also be transmitted by inhalation or swallowing.

Following bacterial, fungal, parasitic or viral infection, an organism's immune defense system is triggered. White blood cells like lymphocytes and monocytes attempt to attack and destroy the invasive microbe/virus. This is part of the body's immune response, which can often lead a patient feeling unwell or fatigued. If a patient's immune system is compromised, or not effective enough to prevent the spread of the infection, severe illness can develop and, in some instances, lead to chronic morbidity and/or death. However, some parasites have the ability to manipulate their host's immune response by producing and releasing immunomodulatory products.

Bacterial infections are commonly treated by antibiotics, which typically act by disturbing the bacterial metabolic processes. However, bacteria mutate easily and antibiotic-resistant strains are beginning to emerge. These resistant strains cause considerably high death rates and increased patient care costs. Broadly speaking, infections caused by resistant bacterial strains lead to up to two-fold higher rates of adverse outcomes compared with similar infections caused by susceptible strains. These adverse outcomes may be clinical (death or treatment failure) or economic (costs of care, length of stay) and reflect both treatment delays and the failure of antibiotic treatment to cure infections. The magnitude of these adverse outcomes will be more pronounced as disease severity, strain virulence, or host vulnerability increase. It is the cost of these treatment delays and failures to patients and the healthcare system that forms the basis of the negative impact of antibiotic resistance (N. D. Friedman, E. Temkin, Y. Carmeli, The negative impact of antibiotic resistance, Clinical Microbiology and Infection, Volume 22, Issue 5, 2016, Pages 416-422).

Another problem is the side effects of many currently used antibiotics, many patients are so sensitive to broad groups of antibiotics (i.e. penicillin) that they could be killed by anaphylactic reactions when using some classes of antibiotics (da Silva A F, Benchimol J L. Malaria and quinine resistance: a medical and scientific issue between Brazil and Germany (1907-19). Med Hist. 2014; 58(1):1).

Fungal diseases are usually treated by subjecting the fungus to a substance which destroys the cell wall. Nevertheless, just as there are antibiotic-resistant bacteria, there are also drug-resistant fungi.

There are several antiparasitic drugs on the market, but the parasites are very diverse biologically and therefore the drugs usually only work on a limited number of parasites. Some antifungal drugs and antibiotics are effective also against parasites. However, for some parasitic infections, no drug is effective and drug resistance for parasites is also a major problem.

While many bacterial and fungal infections can be treated with drugs, there are few drugs that are effective in the treatment of viral infections. Therefore, viral infections are often treated by easing the symptoms while waiting for the infected patient's immune system to kill the virus. Vaccines can prevent the infection of viruses, but these only work prophylactically. Furthermore, viruses can also mutate to gain resistance against existing therapies meaning that treatments only give limited protection in the general population.

Furthermore, current medications against microbial infections (antibiotics, antiviral, antimycotic etc.) tend to be administered so that they are distributed in the entire body even if a higher concentration is required in one specific organ/organs for treatment. Some antibiotics, such as those used to treat urinary tracts infection are concentrated in the urine making them more effective with low systemic toxicity i.e. nitrofurantoin. Additionally, some can be applied by injection to joints or by local dermal application but generally this is not the case and thus is a problem.

Another problem with antibiotics is that many forms have difficulties passing the blood brain barrier.

Unpublished application PCT/EP2019/082800 describes a process for the synthesis of mono- and bis-nitrosylated 1,2-propanediols.

### BRIEF DESCRIPTION OF THE FIGURES

FIG. 1 a) cell viability as a function of PDNO concentration via the MTT assay; b) CPE inhibition as a function of PDNO concentration.

FIG. 2 effect of addition of PDNO on the replication of SARS-CoV-2 in VeroE6 cells as determined by RT-qPCR A) viral replication inhibition (%); B) viral RNA copy number (% of control).

FIG. 3 shows photographs of infected cells treated with 40 μM of PDNO in PD in cell culture medium.

FIG. 4 shows the dose-response relationship between the concentration of PDNO added to schizont stage Dd2 and corresponding parasite viability after 24 h.

FIG. 5 shows the respiratory syncytial virus (RSV) log 10 viral loads (copies/ml) in MucilAir tissues at day 4 after infection and basolateral compartment treatment at day 0 and 2 with PD (solvent control) and 200 μM PDNO or the virus controls (no compound treatment).

### DISCLOSURE OF THE INVENTION

The inventors have surprisingly found that nitrosylated propanediols, in particular mono- and/or bis-nitrosylated propanediols, have antimicrobial, in particular antibacterial, antifungal, antiparasitic and antiviral, properties and can be used to treat an infected patient.

Unless otherwise indicated, all technical and scientific terms used herein have the same meaning as commonly understood by one of ordinary skill in the art to which this invention pertains.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

In particular any embodiments of the medical uses may be combined with the embodiments of the non-aqueous composition. Furthermore, any of the embodiments of the devices may be combined with any of the embodiments of the medical uses and/or non-aqueous composition.

As used herein, the term “comprises” will take its usual meaning in the art, namely indicating that the component includes but is not limited to the relevant features (i.e. including, among other things). As such, the term “comprises” will include references to the component consisting essentially of the relevant substance(s).

As used herein, unless otherwise specified the terms “consists essentially of” and “consisting essentially of” will refer to the relevant component being formed of at least 80% (e.g. at least 85%, at least 90%, or at least 95%, such as at least 99%) of the specified substance(s), according to the relevant measure (e.g. by weight thereof). The terms “consists essentially of” and “consisting essentially of” may be replaced with “consists of” and “consisting of”, respectively.

For the avoidance of doubt, the term “comprises” will also include references to the component “consisting essentially of” (and in particular “consisting of”) the relevant substance(s).

As used herein, the term “microbial infection” refers to a bacterial, viral, fungal or parasitic infection.

### Medical Uses

According to a first aspect of the invention, there is provided a compound of formula (I):

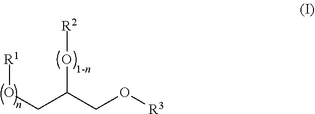

wherein R¹, R² and R³ each independently represent H or —NO,

wherein n is 0 or 1;

wherein when n is 0, R¹ is H; and

wherein when n is 1, R² is H,

provided that at least one of R¹ R² and R³ represents —NO,

for use in the treatment of a microbial infection.

In an alternative first aspect of the invention, there is provided a method of treatment of a microbial infection in a subject, which method comprises administering a therapeutically effective amount of one or more compound of formula (I):

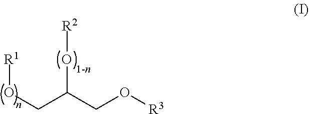

wherein R¹, R² and R³ each independently represent H or —NO,

wherein n is 0 or 1;

wherein when n is 0, R¹ is H; and

wherein when n is 1, R² is H,

provided that at least one of R¹ R² and R³ represents —NO,

to a subject in need of such treatment.

In a further alternative first aspect of the invention, there is also provided the use of a compound of formula (I):

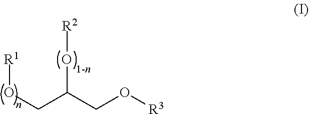

wherein R¹, R² and R³ each independently represent H or —NO,

wherein n is 0 or 1;

wherein when n is 0, R¹ is H; and

wherein when n is 1, R² is H,

provided that at least one of R¹ R² and R³ represents —NO,

for the manufacture of a medicament for treating a microbial infection.

For the avoidance of doubt, references to a microbial infection will include infections of a degree or extent allowing for their identification by a relevant skilled person (e.g. a healthcare practitioner) using techniques known to those skilled in the art.

For example, a bacterial or fungal infection may be diagnosed in a subject/patient by taking a sample from said subject/patient and providing a culture thereof. Similarly, bacterial, fungal and viral infections may be diagnosed by genetic and/or immunological analysis of a sample using routine techniques.

The skilled person will understand that references to treating a subject/patient having a microbial infection do not exclude such a subject/patient being treated for multiple types of such infections, such as two or three such types of infections. For example, compounds of the invention may be used to simultaneously treat a bacterial and viral infection, which treatment may be referred to as treatment of the bacterial infection, treatment of the viral infection or treatment of both infections. Similar reasoning will apply to references to treatment of a general type of infection, such as a viral infection, which will include references to treatment of one or more specific types of infection. Furthermore, references to treatment do not exclude the administration of other anti-microbial treatments, such as antibiotics, either as a co-treatment, or before or after administration of the compound of formula (I).

It is envisaged that due to the broad antimicrobial effects of the compound of formula I, the skilled person need not specifically identify the type of infection before treatment and that the treatment encompasses situations where the exact infectious agent is unknown.

In particular embodiments, the treatment is of a bacterial, fungal or viral infection.

In another embodiment, the treatment is of a parasitic infection.

In particular embodiments, the treatment is of a bacterial infection.

In an embodiment, the bacterial infection is an infection caused by gram-positive bacteria or a gram-negative bacteria.

In the embodiments where the infection is caused by gram-positive bacteria the bacteria may be selected from the phylum Firmicutes; for example the bacteria may be selected from the class Bacilli, such as the bacteria may be selected from the order Bacillales. More specifically the bacteria may be selected from the family Bacillaceae and/or Staphylococcaceae.

In a further embodiment, the bacteria is selected from the genus Bacillus and/or Staphylococcus, such as wherein the bacteria is Bacillus spizizenii (B. Spizizenii) and/or Staphylococcus aureus (S. Aureus).

In the embodiments where the infection is caused by gram-negative bacteria the bacteria may be selected from the phylum Proteobacteria; for example, the bacteria may be selected from the class Gammaproteobacteria, such as the bacteria may be selected from the order Pseudomonadales. More specifically the bacteria may be selected from the family Pseudomonadaceae.

In an embodiment, the bacteria is selected from the genus Pseudomonas, such as wherein the bacteria is Pseudomonas aeruginosa (P. Aeruginosa).

In an embodiment, the treatment is of a urinary tract infection (UTI) caused by a bacteria infection. In a particular embodiment, the treatment is of a catheter-associated urinary tract infection (CAUTI).

In a particular embodiment, the treatment is of a microorganism that has developed resistance to conventional/existing treatments, for example the treatment of resistant strains of Staphylococcus Aureus, such as Methicillin-resistant Staphylococcus Aureus (MRSA).

In an embodiment, the bacteria is selected from the genus Actinobacteria, such as from the family Mycobacteriaceae. Over 190 species are recognized in this genus. This genus includes pathogens known to cause serious diseases in mammals, including in humans. The Greek prefix myco- means “fungus,” alluding to the way mycobacteria have been observed to grow in a mould-like fashion on the surface of cultures. It is acid fast and cannot be stained by the Gram stain procedure.

In a further embodiment, the treatment is of a fungal infection.

In a particular embodiment the fungal infection is caused by fungus selected from the division Ascomycota, such as wherein the fungus is selected from the class Saccharomycetes; for example wherein the fungus is selected from the order Saccharomycetales, optionally wherein the fungus is selected from the family Saccharomycetaceae, more specifically wherein the fungus is selected from the genus Candida, for example wherein the fungus is Candida albicans (C. albicans).

In the embodiments where the fungal infections are caused by fungus selected from the division Ascomycota, the fungus may be selected from the class Eurotiomycetes, such as wherein the fungus is selected from the order Eurotiales, for example wherein the fungus is selected from the family Trichocomaceae, more specifically wherein the fungus is selected from the genus Aspergillus, for example wherein the fungus is Aspergillus brasiliensis (A. brasiliensis).

In a further embodiment, the treatment is of a viral infection.

In another embodiment, the treatment is of a respiratory virus.

In an embodiment the viral infection is caused by viruses selected from the realm Riboviria, such as wherein the virus is selected from the phylum Incertae Sedis; for example wherein the virus is selected from the order Nidovirales, more specifically wherein the virus is selected from the family Coronaviridae, such as wherein the virus is selected from the genus Betacoronavirus, such as wherein the virus is selected from the subgenus Sarbecovirus. In a particular embodiment that may be mentioned, the infection is caused by the severe acute respiratory syndrome coronavirus 2 (SARS-CoV-2) virus.

In another embodiment the virus is selected from the phylum Negarnaviricota, for example wherein the virus is selected from the order Monoegavirales, more specifically wherein the virus is selected from the family Paramyxoviridae, such as wherein the virus is selected from the genus Orthopneumovirus, such as wherein the infection is caused by a respiratory syncytial virus (RSV), which is also commonly referred to as human respiratory syncytial virus (hRSV) and human orthopneumovirus.

In the embodiments where the treatment is of a parasitic infection, the parasite may be a unicellular protozoan parasite, for example a unicellular protozoan parasite from the genus Plasmodium, such as wherein the parasite is Plasmodium Falciparum.

In further embodiments wherein the treatment is of a parasitic infection, the parasite may be a parasite in the class Trematoda, such as a parasitic flatworm (also commonly referred to as flukes), for example a parasitic flatworm from the genus Schistosoma.

In all aspects of the invention, the treatment includes treatment of an infection in a patient caused by the microbe, wherein the patient is administrated an effective amount (which may be referred to as a therapeutically effective amount) of the compound of formula (I).

The skilled person will understand that microbial infections as described herein may be present as systemic or local infections, depending of the nature of the infection and the pathology of the disease. For example, fungal infections may manifest as local infections, with the infection being limited to a particular tissue or organ (e.g. the lungs). Viral and bacterial infections may be systemic in nature (e.g. being detectable in plasma) but may also exhibit localised effects (e.g. in the lungs).

The skilled person will understand that references to the treatment of a condition, e.g. the bacterial, parasitic, fungal or viral infection (or, similarly, to treating that condition), take their normal meanings in the field of medicine. In particular, the terms may refer to achieving a reduction in the severity of one or more signs and/or clinical symptoms associated with the condition.

For example, in the case of a viral infection, the term may refer to achieving reduction in the number of viral particles which have infected the patient (i.e. the viral load), a reduction in the number of new viral particles infecting the patient and/or a reduction in the number of new viral particles produced in the infected patient, which in turn result in a reduction of the severity of the symptoms like fever, nausea, shortness of breath and cough.

Similarly in the case of a bacterial infection, the term may refer to achieving reduction in the number of bacteria cells which have infected the patient, a reduction in the number of new bacteria cells infecting the patient and/or a reduction in the reproduction of the bacteria in the patient, which in turn result in a reduction of the severity of the symptoms like fever, nausea, shortness of breath, cough and any of the other symptoms listed herein of bacterial infections.

Also in the case of a fungal infection, the term may refer to achieving reduction in the number of fungal cells which have infected the patient, a reduction in the number of new fungal cells infecting the patient and/or a reduction in the reproduction of the fungal cells in the patient, which in turn result in a reduction of the severity of the symptoms like a rash, itching and swelling of the skin.

Furthermore, in the case of a parasitic infection, the term may refer to achieving reduction in the number of parasites which have infected the patient, a reduction in the number of new parasites infecting the patient and/or a reduction in the reproduction of the parasites in the patient.

As used herein, the term therapeutically effective amount (and similar terms such as effective amount and the like) will refer to an amount of a compound that confers a therapeutic effect on the patient in need thereof (i.e. results in the treatment of the patient). A therapeutic effect may be observed in a manner that is objective (i.e. measurable by some test or marker) or subjective (i.e. the subject gives an indication of and/or feels an effect). In particular, the effect may be observed (e.g. measured) in a manner that is objective, using appropriate tests as known to those skilled in the art.

By the term “viral load”, this refers to the amount of a specific virus in a test sample taken from a patient. For COVID-19, which is the coronavirus 2019 caused by SARS-CoV-2 virus, this can be detected by a nasopharyngeal swab from the patient. The viral load reflects how well a virus is replicating in an infected person. A high viral load detected in a sample means a large number of virus particles are present in the patient.

By the term “bacterial load”, this refers to the amount of a specific bacteria in a test sample taken from a patient. The test sample can be taken form the blood, skin, urine or any other part of the body which is infected. The bacterial load is usually determined through a microbiological culture which is a method of letting the microorganisms reproduce under controlled conditions in a laboratory. The cell culture is used to determine the quantity and quality of the microbials which have infected the patient from which the sample was taken. The bacterial load reflects how well a bacteria is replicating in an infected person. A high bacterial load detected in a sample means a large number of bacteria cells are present in the patient.

By the term “fungal load”, this refers to the amount of a specific fungus in a test sample taken from a patient. The test sample can be taken form the skin, body discharge or any other part of the body which is infected. The fungal load is usually determined through a microbiological culture, as explained above. The fungal load reflects how well a fungus is replicating in an infected person. A high fungal load detected in a sample means a large number of fungal cells are present in the patient.

As used herein, references to subjects will refer to a living subject being treated, including mammalian (e.g. human) subjects. Such subjects may also be referred to as patients. In particular, the term patient may refer to a human subject. Subjects may also include animals (e.g. mammals), such as household pets (e.g. cats and, in particular, dogs), livestock (e.g., cows, sheep, ducks, chickens, pigs etc.) and horses.

Particular symptoms of a patient suffering from the microbial infection that may be mentioned include those selected from the group consisting of: fever, aching muscles, headache, cough, fatigues, nausea, vomiting, diarrhoea, nasal congestion, pain, tiredness, a sore throat, shortness of breath, increased mucus production, weight loss and/or a rash.

The skilled person will understand that the treatment of microbial infections may act to prevent (or reduce the likelihood or risk of) the patient obtaining a secondary disease/complication. Therefore, secondary diseases/complications are also treated which may be additional microbial infections, inflammation of organs, necrosis and sepsis. Additionally, the treatment of the primary infection may comprise prophylaxis of one or more secondary infections (i.e. the secondary infections are prevented by treating the primary infection.

It is preferred that the compound of formula (I) is administered to patients with one or more underlying medical conditions including chronic (long-term) respiratory diseases (such as asthma, chronic obstructive pulmonary disease (COPD), emphysema or bronchitis), chronic heart disease (such as heart failure), (chronic kidney disease), chronic liver disease (such as hepatitis), chronic neurological conditions (such as Parkinson's disease, motor neuron disease, multiple sclerosis, a learning disability or cerebral palsy), diabetes, problems with your spleen (for example, sickle cell disease or if you have had your spleen removed), a weakened immune system as the result of conditions (such as HIV and AIDS, or medicines such as steroid tablets or chemotherapy), being seriously overweight (a body mass index (BMI) of 40 or above) and/or pregnancy.

The skilled person will understand that compounds of the invention may be administered (for example, as formulations as described hereinabove) at varying doses, with suitable doses being readily determined by one of skill in the art.

In an embodiment the compound of formula (I) may be administered intravenously, intraarterially, intranasally, via inhalation, subcutaneously, via intraarticular injections into infected joints, sublingually, intravesically, dermally, gastrointestinally, vesically or intramuscularly.

The ability for the compound of formula (I) to be administered in many different ways allows for the treatment to be highly organ specific, meaning that for local infections only that organ/part of the body is treated whilst avoiding systemic spread in the entire body, leading to a more efficacious treatment with reduced chances of side effects.

By the term “inhalation” it is envisaged that the compound of formula (I) is inhaled as a vapour or an aerosol through the nose and/or mouth. Furthermore, inhalation may also be through a nasal or tracheal catheter, an endotracheal tube or a supraglottic airway device.

In a particular embodiment, the administration is to a nasal mucous membrane, wherein administration is via applying a gel or liquid directly to the nasal cavity of the patient. In this embodiment, although the compound of formula (I) is administered directly to the mucous membrane in the nasal cavity, through dispersion in the body it is envisaged that the compound of formula (I) reaches other epithelial layers of the patient, in particular the epithelial layers in the mouth, nose, trachea, or lungs of the patient.

In an embodiment, for nasal administration the compound of formula (I) may be applied as a spray or as a gel which is rubbed against the mucosal surface.

In a particular embodiment, the administration is subcutaneous, wherein the administration is via applying a gel or liquid to the cutis or subcutis of the patient. In this embodiment, the compound of formula (I) may be injected to the cutis or subcutis with a syringe.

In a particular embodiment, the administration is intramuscular, wherein the administration is via applying a gel or liquid to a muscle of the patient. In this embodiment, the compound of formula (I) may be injected to the muscle with a syringe. In an embodiment, via intramuscular administration the compound of formula (I) is administered to is a skeletal muscle, smooth muscle or cardiac muscle.

The skilled person will be able to determine a suitable dose of active ingredients to be used in treatment based on the nature of the formulation (e.g. the combination of pharmaceutical formulation and suitable buffer as described herein) used, the condition to be treated and the status (e.g. state of illness) of the patient. For example, when administered intravenously or intraarterially to human adult a suitable dose may be about 0.5 to about 3,000 nmol/kg/min, such as about 1 to about 3,000 nmol/kg/min, for example from about 5 to about 3,000 nmol/kg/min of the compound(s) of formula I. Such doses may be administered by infusion (either continuous or pulsed), such as infusion over an extended period of time (e.g. 1 to 2 hours or even up to one, two or three weeks), or may be administered as a single (bolus) dose (such as a one-off dose or a single dose per treatment intervention, such as a single dose as required, or a single dose in each 24 hour period during treatment).

In an embodiment, where the administration is by subcutaneous injection (e.g. subcutaneous administration), the dose of the compound of formula (I) is in the range of from about 1 to about 30,000 nmol kg⁻¹ min⁻¹, such as from about 100 to about 2000 nmol kg⁻¹ min⁻¹.

In an embodiment, where the administration is by intramuscular injection (e.g. intramuscular administration), the dose of the compound of formula (I) is in the range of from about 1 to about 30,000 nmol kg⁻¹ min⁻¹, such as from about 10 to about 1000 nmol kg⁻¹ min⁻¹.

In an embodiment, where the administration is intranasal, the dose of the compound of formula (I) is in the range of from about 1 to about 30,000 nmol kg⁻¹, such as from about 100 to about 3000 nmol kg⁻¹.

In an embodiment, where the administration is sublingual, the dose of the compound of formula (I) is in the range of from about 1 to about 30,000 nmol kg⁻¹, such as from about 100 to about 3000 nmol kg⁻¹.

In an embodiment, where the administration is dermal, the dose of the compound of formula (I) is in the range of from about 1 to about 50,000 nmol kg⁻¹, for example from about 50 to about 30,000 nmol kg⁻¹ such as from about 100 to about 3000 nmol kg⁻¹.

In an embodiment the compound of formula (I) is administered in an amount of from about 1 μM to about 10,000 μM, such as from about 1 μM to about 1000 μM (e.g. about 1 μM to about 750 μM, for example from about 1 μM to about 500 μM, such as from about 1 μM to about 250 μM, for example about 1 μM to about 100 μM, or about 1 μM to about 50 μM).

Administration of active ingredients may be continuous or intermittent. The mode of administration may also be determined by the timing and frequency of administration but is also dependent on the severity of that condition, or otherwise on the need for treatment.

Preferably the compound of formula (I) is administrated as a substantially non-aqueous composition further comprising a compound of formula (I) but wherein R¹, R² and R³ represent H.

The amount of active ingredient in a composition will depend on the severity of the bacterial, parasitic, fungal or viral infection, and on the subject, to be treated.

In any event, the practitioner, or other skilled person, will be able to determine routinely the actual dosage, which will be most suitable for an individual patient. Dosages mentioned herein are exemplary of the average case; there can, of course, be individual instances where higher or lower dosage ranges are merited, and such are within the scope of this invention.

The skilled person will understand that the temperature at which compounds of formula (I) are administered in treatment (i.e. administered to a subject) may be that of the environment in which administration takes place (i.e. room temperature) or may be controlled. For example, such formulations may be formed and/or administered at room temperature or at a reduced temperature (i.e. a temperature that is below room temperature), such as from about −10° C. to about 25° C., such as from about −5° C. to about 25° C., for example from about 0 to about 25° C.

Without wishing to be bound by theory, it is believed that upon administration to a patient, the compounds of formula (I) are hydrolysed to release nitric oxide, which alone and in combination with the propanediol, which also works as an antimicrobial alone, provides the surprising synergistic antimicrobial effect found by the inventors.

In all aspects of the invention, the treatment reduces or inhibits replication of the microbe in a patient, prevents infection of a patient by the microbe, kills the microbe in a patient and/or affects the immune system of a patient positively.

In an embodiment, the treatment reduces or inhibits replication of the bacteria, fungus and/or virus in a patient, prevents infection of a patient by the bacteria, fungus and/or virus, kills the bacteria, fungus and/or virus in a patient and/or affects the immune system of a patient positively.

In another embodiment, the treatment reduces or inhibits replication of a parasite in a patient and/or prevents infection of a patient by a parasite.

A particular compound of the first aspect of the invention is a compound according to formula (II)

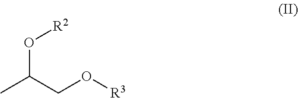

wherein R² and R³ each independently represent H or —NO, provided that at least one of R² and R³ represents —NO.

Two enantiomers of the compound according to formula (II) exist, being the R and S form as depicted below:

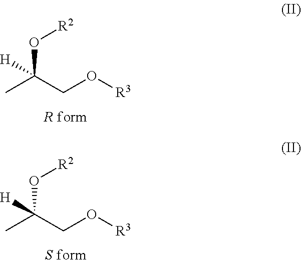

The compounds of formula (I) may contain an asymmetric carbon atom as outlined above and will therefore exhibit optical isomerism.

All stereoisomers and mixtures thereof of the compounds according to formula (I) are included within the scope of the invention.

A further particular compound of the first aspect of the invention is a compound according to formula (III):

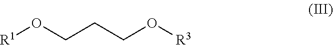

wherein R¹ and R³ each independently represent H or —NO, provided that at least one of R¹ and R³ represents —NO.

A further particular compound of the first aspect of the invention is a compound according to formula (IV):

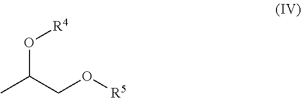

wherein R⁴ and R⁵ each independently represent H or —NO, provided that at least one of R⁴ and R⁵ represents —NO.

As detailed above, in an embodiment the compound of formula (I) is administered as a substantially non-aqueous composition further comprising a compound of formula (I) but wherein R¹, R² and R³ represent H.

As used herein, references to “substantially non-aqueous” will refer to the component comprising less than 1% (such as less than 0.5% or less than 0.1%, e.g. less than 0.05%, less than 0.01%) by weight of water.

Particular substantially non-aqueous compositions of the invention that may be mentioned include those wherein the composition comprises from about 0.01% to about 9% (e.g. about 0.01% to about 5%, such as about 3% to about 5%, or about 5% to about 7%) by weight of the one or more of the compounds of the invention (i.e. compounds of formula I).

Particular substantially non-aqueous compositions of the invention that may be mentioned include those wherein the composition comprises from about 1 to about 1000 mM (e.g. about 5 to about 750 mM, such as about 5 to about 500 mM, or about 10 to about 203 mM) of the one or more of the compounds of the invention (i.e. compounds of formula I).

Further substantially non-aqueous compositions of the invention that may be mentioned include those wherein the composition comprises from about 1 μM to about 10,000 μM, such as from about 1 μM to about 1000 μM (e.g. about 1 μM to about 750 μM, for example from about 1 μM to about 500 μM, such as from about 1 μM to about 250 μM, for example about 1 μM to about 100 μM, or about 1 μM to about 50 μM).

For the avoidance of doubt, the unit mM refers to the concentration of the compound of formula (I) in the non-aqueous composition in 10⁻³ mol/L and, where the composition comprises a mixture of compounds of formula I, is based on the average molecular weight of the compounds of formula I in the composition.

For the avoidance of doubt, the unit μM refers to the concentration of the compound of formula (I) in the non-aqueous composition in 10⁻⁶ mol/L and, where the composition comprises a mixture of compounds of formula I, is based on the average molecular weight of the compounds of formula I in the composition.

Particular substantially non-aqueous compositions of the invention that may be mentioned include those wherein the composition comprises a compound according to formula (II). Preferably the compound according to formula (II) is the S form.

The S form of the compound according to formula (II) is preferred as this has a higher rate of metabolism than the R form. Furthermore, the S form has a different metabolic degradation route, which results in metabolites which are less toxic than those from the R form.

Particular substantially non-aqueous compositions of the invention that may be mentioned include those wherein the composition comprises a compound according to formula (III).

Preferably the compound according to formula (II) is the S form, although it is envisaged that the product is a mixture of both the S and R form of formula (II) with the S form preferably being present in an enantiomeric excess (ee).

In particular embodiments, the compound according to formula (II) may be in an enantiomeric excess of the S form of the compound. That is to say, greater than 50 ee % of the product is in the S form, such as greater than, or equal to, 60 ee %, 70 ee %, 80 ee %, 90 ee %, 95 ee % or 98 ee % of the product is the S form.

In an embodiment where the product is a mono-nitrosylated compound according to formula (II), greater than 50 wt. % of the product is nitrosylated in the 2 position (i.e. R² is —NO), such as between about 55 wt. % and about 80 wt. % is nitrosylated in the 2 position, for example between about 55 wt. % and 75 wt. %.

Particular substantially non-aqueous compositions that may be mentioned include those wherein the composition consists essentially of one or more compounds of formula I and corresponding compounds of formula I but wherein R¹, R² and R³ represent H (i.e. 1,2-propanediol and/or 1,3-propanediol).

Other particular substantially non-aqueous compositions may comprise (or, particularly, consist essentially of or, more particularly, consist of) one or more compounds of formula II and 1,2-propanediol.

Equally, further substantially non-aqueous compositions may comprise (or, particularly, consist essentially of or, more particularly, consist of) one or more compounds of formula III and 1,3-propanediol.

By the term “consist essentially of”, this means that at least 90 wt. % of the defined feature is present, such as at least 95 wt. %, 96 wt. %, 97 wt. %, 98 wt. % or 99 wt. % of the defined feature is present.

Furthermore, particular substantially non-aqueous compositions that may be mentioned include those wherein the composition comprises (or, particularly, consists essentially of or, more particularly, consists of) one or more compounds of formula (II) and (III) along with 1,2-propanediol and 1,3-propanediol.

Particular substantially non-aqueous compositions that may be mentioned include those wherein the composition is substantially free of dissolved nitric oxide.

By the term “substantially free”, this means that the non-aqueous compositions of the invention comprise less than 5 wt. %, 4 wt. %, 3 wt. %, 2 wt. % or 1 wt. % of dissolved nitric oxide, such as less than 0.5 wt. % or 0.1 wt. %.

Furthermore, particular substantially non-aqueous compositions may comprise:

(a) one or more compounds of formula IV

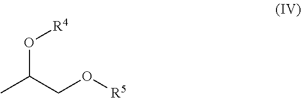

wherein R⁴ and R⁵ each independently represent H or —NO, provided that at least one of R⁴ and R⁵ represents —NO; and

(b) 1,2-propanediol.

The substantially non-aqueous compositions may be administered alone or may be administered by way of known pharmaceutical compositions/formulations.

Accordingly, the substantially non-aqueous composition may be comprised in a pharmaceutical formulation, optionally wherein the pharmaceutical formulation comprises one or more pharmaceutically acceptable excipients.

The skilled person will understand that references herein to pharmaceutical formulations herein refer to the substantially non-aqueous composition in the form of a pharmaceutical formulation and will include references to all embodiments and particular forms thereof.

As used herein, the term pharmaceutically-acceptable excipients includes references to vehicles, adjuvants, carriers, diluents, pH adjusting and buffering agents, tonicity adjusting agents, stabilizers, permeability enhancers, wetting agents and the like. In particular, such excipients may include adjuvants, diluents or carriers.

Particular pharmaceutical formulations that may be mentioned include those wherein the pharmaceutical formulation comprises at least one pharmaceutically acceptable excipient.

Particular pharmaceutical formulations that may be mentioned include those wherein the one or more pharmaceutically acceptable excipients are substantially non-aqueous.

For the avoidance of doubt, references herein to compounds of formula (I) for particular uses may also apply to compositions and pharmaceutical formulations comprising compounds of the invention, as described herein.

In an alternative embodiment the compound of formula (I) is administered as an aqueous composition further comprising a compound of formula (I) but wherein R¹, R² and R³ represent H. Particular aqueous compositions that may be mentioned include those wherein the composition comprises a compound of formula (I) and 1,2-propanediol and/or 1,3-propanediol, wherein the compound of formula (I) and 1,2-propanediol and/or 1,3-propanediol are present in the composition in a concentration of 0.1 to 1 vol-%, such as from 0.3 to 0.8 vol-%, for example 0.5 vol-%.

### Processes for Preparing the Compounds of Formula (I)

Also described herein is a process for the preparation of a composition comprising one or more compounds of formula I

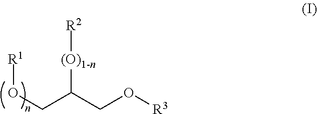

wherein:

R¹, R² and R³ each independently represent H or —NO;

n is 0 or 1;

wherein when n is 0 then R¹ is H, and when n is 1 the R² is H; and

provided that at least one of R¹ R² and R³ represents —NO,

said process comprising the step of:

(i) reacting a corresponding compound of formula I but wherein R¹, R² and R³ represent H with a source of nitrite, optionally in the presence of a suitable acid,

wherein:

(a) when the source of nitrite is an organic nitrite, step (i) is performed in a suitable organic solvent; and

(b) when the source of nitrite is an inorganic nitrite, step (i) is performed in a bi-phasic solvent mixture comprising an aqueous phase and a non-aqueous phase.

For the avoidance of doubt, the product of the process (i.e. the compound of formula I) may also (or instead) be referred to as a mono- and bis-nitrosylated 1,2-propanediol or 1,3-propanediol (or a mixture of such compounds, i.e. a composition comprising one or more mono- or bis-nitrosylated 1,2- or 1,3-propanediol).

For the avoidance of doubt, the corresponding compound of formula I, but wherein R¹, R² and R³ represent H, may be referred to as a corresponding 1,2-propanediol and/or 1,3-propanediol (i.e. corresponding to the structure of the desired product), which may in turn be referred to as the starting material for the process of the invention. Put another way, the corresponding compound of formula I may be a compound according to formula (Ia) as defined below.

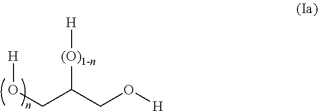

For the avoidance of doubt, where the integer (n or 1-n) as relating to the oxygen atoms is 0, no oxygen atom is present and the substituent R¹ and R² (and the corresponding H in the compound of formula (Ia)) is bonded to the respective carbon.

The skilled person will understand that references herein to the process will include references to all embodiments and particular features thereof.

The skilled person will understand that references to the preparation of a composition comprising one or more compounds of formula (I) will refer to the preparation of a composition that contains, as a constituent part, an amount of one or more compounds the structure of which is as defined in formula I, optionally together with other compounds. The process may also be referred to a process for preparing compounds of formula I (i.e. a process for preparing one or more compounds of formula I).

The skilled person will understand that references to the process being a process for preparing compounds of formula I will be understood to indicate that the process may result in the preparation of one or more types of compound each as described by formula I as defined herein (e.g. where more than one such compound is present, as a mixture thereof).

As such, the skilled person will also understand that the compounds formed in the process may take the form of a mixture of each mono-nitrite and the di-nitrite products, with the relative amounts of each varying depending on the concentration of compounds of formula I.

In particular, the process may allow for the preparation of a composition wherein at least 50 wt. %, 60 wt. %, 70 wt. % or 80 wt. % (such as at least 90 wt. % or at least 99 wt. %, e.g. at least 99.9 wt. %) of the compounds of formula I are mono-nitrosylated, such that R¹, R² and R³ each independently represent H or —NO, provided that one of R¹, R² or R³ represents —NO and the other groups represent H.

In particular, the process may result in the preparation of the composition that comprises one or more compounds of formula I together with one or more corresponding compounds of formula I but wherein R¹, R² and R³ represent H (i.e. 1,2-propanediol and/or 1,3-propanediol, e.g. unreacted 1,2-propanediol and/or 1,3-propanediol starting material), and optionally other compounds.

In certain embodiments, the process may be a process for preparing a composition consisting essentially of one or more compounds of formula I, and one or more corresponding compounds of formula I but wherein R¹, R² and R³ represent H (i.e. 1,2-propanediol and/or 1,3-propanediol; e.g. as a mixture thereof).

The skilled person will understand that the term “reacting” will refer to bringing the relevant components together in a manner (e.g. in suitable state and medium) such that a chemical reaction occurs. In particular, the reference to reacting the starting material (i.e. 1,2-propanediol and/or 1,3-propanediol) with a source of nitrite will refer to a chemical reaction between the starting material and the nitrite (i.e. the nitrite provided by the source of nitrite).

The skilled person will understand that the reference to “a source of nitrite” may instead refer simply to “nitrite”, as it is the nitrite provided by the source of nitrite which undergoes chemical reaction. As such, references to a source of nitrite will be understood to refer to a compound that provides, for reaction, a nitrite moiety (which may be present either in ionic or covalently bonded form, depending on the source of nitrite present). The source of nitrite may therefore be referred to as a source of reactive (or reactable) nitrite (or nitrite moiety). For the avoidance of doubt, the source of nitrite may be an inorganic nitrite or an organic nitrite.

As indicated herein, when the source of nitrite is an organic nitrite, step (i) is performed in a suitable organic solvent.

The skilled person will understand that various organic nitrites may be used in the process of the invention, such as alkyl nitrites.

Particular alkyl nitrites that may be mentioned include ethyl nitrite, propyl nitrites, butyl nitrites and pentyl nitrites. In particular embodiments, the alkyl nitrite is n-butyl nitrite, isobutyl nitrite or tert-butyl nitrite, such as tert-butyl nitrite.

Where the source of nitrite is an organic nitrite, the skilled person will be able to select a suitable solvent. For example, suitable solvents may include those referred to herein as suitable organic components of a biphasic solvent system, and mixtures thereof.

For the avoidance of doubt, unless specified otherwise, the references to the process of the invention being performed in a suitable organic solvent do not indicate that other non-organic solvents, such as water, may be present.

In a particular embodiment, where the process is performed in a suitable organic solvent, the solvent may be essentially water free (which may be referred to as a being “water free” or “dry”), which may indicate that the solvent contains less than about 1% (e.g. less than about 0.1%, such as less than about 0.01%) by weight of water.

The term “about” is defined, herein, as meaning that the defined value may deviate by ±10%, such as by ±5%, for example by ±4%, ±3%, ±2%, or ±1%. The term “about” can be removed from throughout the specification without departing from the teaching of the invention.

As indicated herein, when the source of nitrite is an inorganic nitrite, step (i) is performed in a bi-phasic solvent mixture comprising an aqueous phase and a non-aqueous phase.

The skilled person will understand that the term “bi-phasic solvent mixture” as used herein will refer to a system comprised of two solvents or solvent mixtures which do not mix to form a single solvent phase but instead are present as two distinct (i.e. non-mixed) phases.

Where such solvent mixtures comprise water and an organic solvent (or mixture of organic solvents) such solvent systems may be said to comprise an “aqueous phase” and an “organic phase”. For the avoidance of doubt, the term bi-phasic does not indicate that substances forming other phases, such as substances forming a solid phase, may be present in addition to the solvent system (that is to say, other phases may also be present).

Particular sources of inorganic nitrites that may be mentioned include metal nitrites, such as alkali metal nitrites and alkaline earth metal nitrite. Ionic liquids may also be a suitable source of inorganic nitrites.

For the avoidance of doubt, the term alkali metal takes its usual meaning in the art, namely referring to IUPAC group 1 elements and cations, including lithium, sodium, potassium, rubidium, caesium and francium.

For the avoidance of doubt, the term alkaline earth metal takes its usual meaning in the art, namely referring to IUPAC group 2 elements and cations, including beryllium, magnesium, calcium, strontium, barium and radium.

More particular inorganic nitrites that may be mentioned include alkali metal nitrites, such as lithium nitrite, sodium nitrite and potassium nitrite. In a particular embodiment, the source of nitrite is sodium nitrite.

Alternatively, the metal nitrite may be an alkaline earth metal nitrite, such as lithium nitrite, magnesium nitrite or calcium nitrite.

For the avoidance of doubt, the skilled person will understand that the non-aqueous phase in the bi-phasic solvent system may be an organic solvent, which may therefore be referred to as an organic phase.

The skilled person will be able to select a suitable non-aqueous (i.e. organic) solvent based on the properties of the aqueous phases. For example, where the aqueous phase has a certain level of substances dissolved therein (e.g. ionic solids, such as salts), a wide-range of organic solvents may be selected in order to form a bi-phasic solvent system.

In particular embodiments, the non-aqueous phase consists of a water immiscible organic solvent. In more particular embodiments, the water immiscible organic solvent is an aprotic organic solvent.

Particular water immiscible organic solvents (i.e. particular solvents forming the non-aqueous phase) that may be mentioned include ethers (e.g. tert-butyl methyl ether, cyclopentyl methyl ether, methyl tetrahydrofuran, diethyl ether, diisopropyl ether) and dichloromethane (DCM).

More particular water immiscible organic solvents (i.e. particular solvents forming the non-aqueous phase) that may be mentioned include dichloromethane, diethyl ether and tert-butyl methyl ether. In more particular embodiments, the water immiscible organic solvent is tert-butyl methyl ether.

In certain embodiments that may be mentioned, the solvent mixture may comprise excess compounds of formula I but wherein R¹, R² and R³ represent H (i.e. 1,2-propanediol and/or 1,3-propanediol). For the avoidance of doubt, in such circumstances, the 1,2-propanediol and/or 1,3-propanediol (i.e. the compounds of formula I but wherein R¹, R² and R³ represent H) may be present as both a solvent (e.g. a component of a solvent mixture) and a reagent. As such, in particular embodiments, the process is a process for preparing compounds of formula I as a solution in corresponding compounds of formula I but wherein R¹, R² and R³ represent H, i.e. 1,2-propanediol and/or 1,3-propanediol (e.g. in the form of a mixture comprising 1,2-propanediol and/or 1,3-propanediol, as appropriate). In certain embodiments, when the source of nitrite is an organic nitrite, the solvent may consist essentially of compounds of formula I but wherein R¹, R² and R³ represent H (i.e. 1,2-propanediol and/or 1,3-propanediol). That is to say, the compounds of formula I but wherein R¹, R² and R³ represent H may act both as solvent and as reactant.

In an alternative embodiment, when the source of nitrite is an inorganic nitrite, step (i) may be performed in a single solvent, wherein the solvent may consist essentially of compounds of formula I but wherein R¹, R² and R³ represent H (i.e. 1,2-propanediol and/or 1,3-propanediol). That is to say, the compounds of formula I but wherein R¹, R² and R³ represent H may act both as solvent and as reactant.

In alternative embodiments, the process of the invention may be performed with an excess of nitrite relative to the starting material of formula I but wherein R¹, R² and R³ represent H (i.e. 1,2-propanediol and/or 1,3-propanediol).

As used herein, the term “excess” will take its usual meaning in the art, namely indicating that the component is present in a greater than stoichiometric amount for the reaction in which it is a reagent.

As indicated herein, the process (in particular, the reaction between components) is optionally performed in the presence of a suitable acid.

Particular processes that may be mentioned include those wherein the step of reacting the starting material (i.e. 1,2-propanediol and/or 1,3-propanediol) with a source of nitrite is carried out in the presence of a suitable acid.

Particular acids that may be mentioned as suitable acids include Brønsted acids (i.e. proton donor acids), more particularly wherein such acids may be referred to as a strong acid.

For the avoidance of doubt, the term “strong acid” takes its usual meaning in the art, referring to Brønsted acids whose dissociation is substantially complete in aqueous solution at equilibrium. In particular, references to strong acids may refer to Brønsted acids with a pKa (in water) of less than about 5 (for example, less than about 4.8). For the avoidance of doubt, for multiprotic acids, such as sulphuric acid, the term strong acid refers to the dissociation of the first proton.

Certain strong acids that may be mentioned include those with a pKa (in water) of less than about 1, such as less than about 0 (e.g. less than about −1 or -2). For example, strong acids that may be mentioned include those with a pKa (in water) of about −3. The skilled person will understand that suitable acids may include non-nucleophilic acids, as known to those skilled in the art.

Particular suitable acids that may be mentioned include sulphuric acid, phosphoric acid, trifluoroacetic acid and acetic acid.

More particular suitable acids that may be mentioned include mineral acids (e.g. strong mineral acids), such as sulphuric acid.

The skilled person will be able to select suitable amounts of reagents to use in the process within the teaching herein. For example, the ratio (i.e. the molar ratio) of corresponding compound of formula I but wherein R¹, R² and R³ represent H to nitrite to acid (where present) may be about 1:from about 1 to about 5:to about 0.5 to about 3.5, for example about 1:from about 1 to about 3:from about 0.5 to about 2 (such as about 1:4:2.7, or about 1:2:0.95, or about 1:2:1). For the avoidance of doubt, where a suitable acid is not present, the ratios between the corresponding compound of formula I but wherein R¹, R² and R³ represent H and nitrite may still apply.

In particular embodiments, process step (i) is carried out at a temperature of from about −30° C. to about 5° C., such as from about −30° C. to about 0° C., for example from about −30° C. to about −10° C., preferably from about −25° C. to about −15° C.

In particular embodiments, process step (i) is carried out under an inert atmosphere, such as a nitrogen or argon atmosphere, preferably an argon atmosphere. Furthermore, in particular embodiments any steps of the process may be carried out under an inert atmosphere, such as a nitrogen or argon atmosphere, preferably an argon atmosphere.

Particular processes that may be mentioned, particularly in which a bi-phasic solvent system is used, include those wherein the process further comprises, after (e.g. directly following) step (i), the step of:

(ii) removing substantially all of the aqueous phase (i.e. removing substantially all water) from the solvent mixture.

The skilled person will appreciate that the aqueous phase may be removed from the solvent mixture by any suitable process and using any suitable equipment as known in the art (for example, by using a separating funnel or similar apparatus).

As used herein, unless otherwise specified the term “substantially all” will refer to at least 80% (e.g. at least 85%, at least 90%, or at least 95%, such as at least 99%) of the specified substance(s), according to the relevant measure (e.g. by weight thereof).

The skilled person will also understand that references to “removing substantially all of the aqueous phase from the solvent mixture” may be replaced with references to “removing some or all of the aqueous phase from the solvent mixture” or simply “removing the aqueous phase from the solvent mixture”.

For the avoidance of doubt, in the context of its removal, the term aqueous phase will refer to the (separate) phase formed from water and components dissolved therein.

Particular processes that may be mentioned, particularly in which a bi-phasic solvent system is used, include those wherein the process further comprises, after (e.g. directly following) step (i), the steps of (in the sequence shown):

(ii) removing some or all (e.g. substantially all) of the aqueous phase (i.e. of water);

(iii) washing the remaining organic phase with one or more further aqueous phase;

(iv) optionally repeating steps (ii) and (iii) one or more times.

Further processes that may be mentioned, particularly in which a bi-phasic solvent system is used, include those wherein the process further comprises, after (e.g. directly following) step (i), the steps of (in the sequence shown):

(ii) removing some or all (e.g. substantially all) of the aqueous phase (i.e. of water);

(iii) washing the remaining organic phase with one or more further aqueous phase;

(iv) optionally repeating steps (ii) and (iii) one or more times;

(v) optionally reducing (i.e. reducing the amount/volume of) the organic phase, such as by removal some or substantially all of the water immiscible organic solvent (e.g. organic solvent other than 1,2 propanediol and/or 1,3-propanediol), and

(vi) optionally drying the product,

wherein steps (ii) to (vi) may be performed in any order provided that steps (ii) to (iv) are performed before steps (v) and (vi).

In particular embodiments, process steps (ii) to (iv) may be carried out at a temperature of from about −20° C. to about 5° C., such as from about −10° C. to about 5° C.

In particular embodiments, process step (v) may be carried out at a temperature of from about 0° C. to about 30° C., such as from about 10° C. to about 30° C., for example from about 15° C. to about 30° C.

In particular embodiments, process step (v) is carried out for no more than 6 hours, for example no more than 5 hours, preferably no more than 4 hours.

In particular embodiments, each of steps (ii) to (vi) are performed, such as wherein those steps are performed in the order indicated.

For the avoidance of doubt, the skilled person will understand that washing the remaining organic phase with one or more further aqueous phase will refer to steps comprising: adding a further portion of aqueous solvent (e.g. water); mixing with the (separate) organic phase (e.g. by stirring and/or shaking together); and removing substantially all of the aqueous phase, and optionally repeating said steps one or more times.

The skilled person will understand that step (iii) may be performed by any suitable process and using any suitable equipment known in the art (for example, using a separating funnel).

The skilled person will understand that step (v) may be performed by any suitable process and using any suitable equipment known in the art (for example, by evaporation under reduced pressure).

In the context of step (v), references to removal of the some of the organic phase may refer in particular to removal of substantially all of the water immiscible organic solvent, as defined herein. More particularly, removal of the water immiscible organic solvent may refer to removal of at least 99% (such as at least 99.5%, 99.9% or, in particular, 99.99%) by weight of the water immiscible organic solvent.

Such removal of the water immiscible organic solvent may also refer to removal such that the product following such removal contains less than 1% (such as less than 0.5%, 0.1%, e.g. less than 0.05%, less than 0.01%) by weight of the water immiscible organic solvent.

For the avoidance of doubt, in the context of step (v), references to removal of the organic phase, such as the water immiscible organic solvent, will refer to the removal of any such solvents as defined herein (e.g. the removal of dichloromethane or tert-butyl methyl ether). Where further organic solvents are present (such as those which are not water immiscible, e.g. excess 1,2-propanediol and/or 1,3-propanediol acting as a solvent) a portion of such solvents may be also removed (e.g. together with a water immiscible organic solvent).

In the context of steps (vi), references to drying the product will refer to the removal of water from the material remaining after preceding steps. Such removal of water may refer to removal such that the product following such drying contains less than 1% (such as less than 0.5% or less than 0.1%, e.g. less than 0.05% or less than 0.01%) by weight of water.

The skilled person will understand that step (vi) may be performed by any suitable process and using any suitable equipment known in the art (for example, by contacting the remaining organic phase with a suitable drying agent, such as anhydrous sodium sulphate, anhydrous magnesium sulphate and/or molecular sieves).

Particular processes that may be mentioned include those wherein the process further comprises the step (e.g. after step (i) and, if present, other steps as described herein) of adding a further amount of corresponding compound of formula I but wherein R¹, R² and R³ represent H (i.e. 1,2-propanediol and/or 1,3-propanediol), such that the combined mixture of the one or more compounds of formula I and corresponding compounds of formula I but wherein R¹, R² and R³ represent H (i.e. 1,2-propanediol and/or 1,3-propanediol) comprises from about 0.01% to about 9% (e.g. about 0.01% to about 5%, such as about 3% to about 5%, or about 5% to about 7%) by weight of the one or more of the compounds of the invention.

As outlined above, all embodiments of the process and particular features mentioned herein may be taken in isolation or in combination with any other embodiments and/or particular features mentioned herein (hence describing more particular embodiments and particular features as disclosed herein) without departing from the disclosure of the process.

For example: the process step (i) being carried out at a temperature of from about −30° C. to about 5° C. may be combined with the feature of the process steps (ii) to (iv) may be carried out at a temperature of from about −20° C. to about 5° C.; the feature of the process step (v) being carried out at a temperature of from about 0° C. to about 30° C.; and/or the feature of process step (v) being carried out for no more than 6 hours.

More particular processes that may be mentioned include those wherein the parameters specified are in accordance with the examples provided herein.

A particular product of the process is a compound according to formula (II)

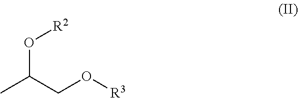

wherein R² and R³ each independently represent H or —NO, provided that at least one of R² and R³ represents —NO, wherein the process comprises the step of reacting 1,2-propanediol (i.e. the starting material) with a source of nitrite, under conditions as described herein (including all embodiments thereof).

Two enantiomers of the compound according to formula (II) exist, being the R and S form as depicted below:

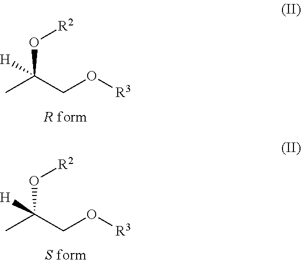

A further particular product of the process is a compound according to formula (III) as depicted below:

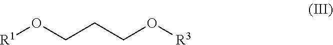

wherein R¹ and R³ each independently represent H or —NO, provided that at least one of R¹ and R³ represents —NO, wherein the process comprises the step of reacting 1,3-propanediol with a source of nitrite.

The two particular processes depicted above for the production of compounds according to formula (II) and (III) may be carried out together or independently of one another.

Based on the occurring biphasic nature of the reaction mixture, optional addition of a phase-transfer catalyst (PTC) may support the product formation. Common PTCs are for example, but not limited to, tetraalkylammonium ions, such as Me₄N+, Et₄N+, Bu₄N+, or Bu₃(N+)CH₂PHCl, with counterions such as ═Cl—, Br—, HSO₄—, or other types of alkylammonium PTCs such as Aliquat® 336, in substoichiometric amounts of <1 equivalent, for example, but not exclusively, in the range of about 0.05 to about 40 mol %, such as about 0.1 to about 30 mol %, for example of about 0.1 to about 20 mol %.

A further particular product of the process is a compound according to formula (IV) as depicted below

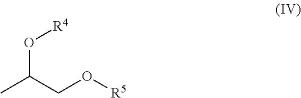

wherein R⁴ and R⁵ each independently represent H or —NO, provided that at least one of R⁴ and R⁵ represents —NO.

A particular process, therefore, is for the preparation of a composition comprising one or more compounds of formula (IV)

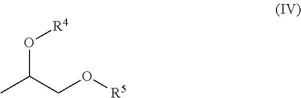

wherein R⁴ and R⁵ each independently represent H or —NO, provided that at least one of R⁴ and R⁵ represents —NO,

said process comprising the step of:

(i) reacting 1,2-propanediol with a source of nitrite, optionally in the presence of a suitable acid,

wherein:

(a) when the source of nitrite is an organic nitrite, step (i) is performed in a suitable organic solvent; and

(b) when the source of nitrite is an inorganic nitrite, step (i) is performed in a bi-phasic solvent mixture comprising an aqueous phase and a non-aqueous phase.

Any of the process steps outlined herein may be combined with the particular process described above with respect to formula (IV) and particular embodiments are outlined below.

In a particular process the inorganic nitrite is a metal nitrite, optionally wherein the metal nitrite is an alkali metal nitrite or an alkaline earth metal nitrite, preferably an alkali metal nitrite.

In a particular embodiment the alkali metal nitrite is sodium nitrite.

In a further particular embodiment the organic nitrite is an alkyl nitrite, such as tert-butyl nitrite.

In a particular process the suitable acid is a strong acid, such as a strong mineral acid (e.g. sulphuric acid).

In a particular embodiment the non-aqueous phase comprises a water immiscible organic solvent, such as a water immiscible aprotic organic solvent.

In an embodiment the water immiscible organic solvent is dichloromethane.

In a particular process, the solvent mixture further comprises excess 1,2-propanediol.

In a further particular process, after step (i) the process further comprises the step of:
(ii) removing substantially all of the aqueous phase from the solvent mixture.

In an embodiment, after step (i) the process further comprises the step(s) of:
(ii) removing some or all (e.g. substantially all) of the aqueous phase (i.e. of water);
(iii) washing the remaining organic phase with one or more further aqueous phase;
(iv) optionally repeating steps (ii) and (iii) one or more times;
(v) optionally reducing (i.e. reducing the amount/volume of) the organic phase, and
(vi) optionally drying the product,

wherein steps (ii) to (vi) may be performed in any order provided that steps (ii) to (iv) are performed before steps (v) and (vi).

In a particular embodiment, the process further comprises the step of adding a further amount of 1,2-propanediol, such that the combined mixture of the one or more compounds of formula I and 1,2-propanediol comprises from about 0.01% to about 9% by weight of the one or more compounds of formula IV.

In particular embodiments of all aspects of the invention as described herein (including all embodiments and combinations of embodiments thereof), the compounds of the invention (e.g. compounds of formula (I)) are prepared using a process as described herein (including all embodiments thereof). For example, in particular embodiments of the first aspect of the invention, the compound of formula (I) is prepared by any one of the processes defined above.

The process for the preparation of the compound(s) of formula (I) as detailed above provides a relatively high concentration of the compounds of the invention in solution, thereby providing ease of handling and minimising storage volumes and transportation costs. Furthermore, the process does not result in dissolved nitric oxide gas or inorganic nitrite, thereby minimising the risk of sudden and spontaneous decomposition, and reducing the potential for side effects when the product of the process is used in therapy. The process also results in only very low levels of other impurities being produced.

Furthermore, the process may deliver chemically stable, non-aqueous compositions and formulations comprising these compounds, which may allow for convenient transport and storage prior to therapeutic use.

In the process to prepare the compounds, the various stereoisomers may be isolated by separation of a racemic or other mixture of the compounds using convention, e.g. fractional crystallisation or HPLC, techniques. Alternatively, the desired optical isomers may be made by reaction of the appropriate optically active starting materials under conditions which will not cause racemisation (i.e. a ‘chiral pool’ method), by reaction of the appropriate starting material with a ‘chiral auxiliary’ which can be subsequently removed at a suitable stage, by derivatisation (i.e. a resolution, including dynamic resolution); for example, with a homochiral acid followed by separation of the diastereomeric derivatives by convention means such as chromatography, or by reaction with an appropriate chiral reagent or chiral catalyst under conditions known to the skilled person.

### EXAMPLES

The invention is illustrated by way of the following examples, which are not intended to be limiting on the general scope of the invention.

### Synthesis Examples

### Abbreviations

aq aqueous

conc concentration

GC gas chromatography

NMR nuclear magnetic resonance

equiv. equivalent(s)

rel. vol. relative volume(s)

For the avoidance of doubt, compounds of formula (I) may also be referred to herein as compounds of the invention and may be referred to by the acronym PDNO, which will indicate that such compounds, including all embodiments and particular features thereof, are used in the methods and uses as described in relation to the present invention. Furthermore, when compositions of PDNO are described that also contain PD, the PD refers to the corresponding propanediol to the compound of formula (I), that is to say the PD is the same compound according to formula (I), but wherein but wherein R¹, R² and R³ represent H.

However, in the context of the examples below, the term “PDNO” specifically refers to compounds according to Formula (II) or (IV). In conjunction with this, the term “PD” refers specifically to 1,2-propanediol, being the starting material from which PDNO is prepared.

### General Procedures

Starting materials and chemical reagents specified in the preparations described below are commercially available from a number of suppliers, such as Sigma Aldrich.

All NMR experiments were performed at 298K on a Bruker 500 MHz AVI instrument equipped with a QNP probe-head with Z-gradients using the Bruker Topspin 2.1 software. Signals were referenced to residual CHCl₃ at 7.27 ppm, unless stated otherwise.

### Stability Assays

Assays of the stability samples were performed by GC/FID, under the following conditions. 1,4-Dioxane was used as the Internal Standard (IS; approximately 0.50 mg/ml in CH₃CN).

GC column: Rxi-5Sil MS, 20 m×0.18 mm, 0.72 μm

Carrier gas: Helium

Inlet: 200° C., split ratio 30:1

Constant flow: 1.0 ml/min

Oven temperature profile: 40° C. (3 min), 10° C./min, 250° C. (3 min)

FID: temp 300° C.; H₂ flow 30 ml/min, Air flow 400 ml/min, make-up flow (N₂) 25 ml/min

### Synthesis Example 1—Preparation of 1-(nitrosooxy)-propan-2-ol, 2-(nitrosooxy)-propan-1-ol and 1,2-bis(nitrosooxy)propane with Sodium Nitrite

1,2-propanediol (15 mL, 205 mmol), water (100 mL), dichloromethane (200 mL) and sodium nitrite (57 g, 826 mmol) were added to a 500 mL three-necked round bottom flask. The mixture was cooled down to 0° C. with an ice bath. Concentrated sulphuric acid (30 mL, 546 mmol) and water (30 mL) were added to a dropping funnel and cooled to 5° C. in a refrigerator. The funnel was adapted to the round bottom flask and the acid added to the nitrite mixture during two hours. The mixture was stirred with a magnet for 20 minutes and then poured into a separation funnel together with more dichloromethane (100 mL) and water (100 mL). The organic phase was separated and dried with sodium sulphate, and reduced on a rotavapor to yield a mixture of 1,2-propanediol (3 wt. %), 1-(nitrosooxy)-propan-2-ol (23 wt. %) 2-(nitrosooxy)-propan-1-ol (13 wt. %) and 1,2-bis(nitrosooxy)propane (57 wt. %).

### Synthesis Example 2—Preparation of 1-(nitrosooxy)-propan-2-ol, 2-(nitrosooxy)-propan-1-ol and 1,2-bis(nitrosooxy)propane with Sodium Nitrite

1,2-propandiol (20 mL, 273.4 mmol), water (60 mL), dichloromethane (120 ml) and sodium nitrite (37.72 g, 546.7 mmol) were added to a 0.5 reactor fitted with a stirrer and flushed with nitrogen and kept during the course of the following reaction under nitrogen. The mixture was cooled down to below 5° C. by cooling the mantle to 0° C. Concentrated sulphuric acid (26.3 g, 260.1 mmol) and water were added to a dropping funnel. The funnel was attached (to the reactor and the acid was added to the nitrite mixture during 33 minutes. The mixture was stirred for 54 minutes and then poured into a flask containing an aqueous saturated sodium bicarbonate solution (100 mL). The mixture was transferred to a separation funnel and the organic phase was washed. The aqueous phase was discarded, and the organic phase was washed with additional aqueous saturated sodium bicarbonate solution (100 mL). The organic phase was dried with magnesium sulphate and then transferred to a 1 L round bottom flask together with 1,2-propandiol (120 ml, 1640 mmol). The solution was reduced on a rotavapor under reduced pressure until the dichloromethane was removed. The removal of dichloromethane was monitored by NMR. A clear solution (134 g) containing 1,2-propandiol (82.8 wt. %), 1-(nitrosooxy)-propan-2-ol (10.4 wt. %), 2-nitrosooxy)-propan-1-ol (6 wt. %) and 1,2-bis(nitrosooxy)propane (0.8 wt. %) was obtained.

¹H-NMR, δ ppm: 5.61 (br s 1H), 4.75-5.58 (m, 2H), 4.11 (br s, 1H), 3.90-3.87 (m, 1H), 3.83-3.69 (m, 2H), 3.60 (dd, J=3.0, 11.2 Hz, 1H), 3.38 (dd, J=7.9, 11.2 Hz, 1H), 1.47 (d, J=6.6 Hz, 3H), 1.39 (d, J=6.4 Hz, 3H), 1.26 (d, J=6.4 Hz, 3H), 1.15 (d, J=6.3 Hz, 3H), Signals for CH and CH₂ of the 1,2-bis(nitrosooxy)propane were below the detection limit.

### Synthesis Example 3—Preparation of 1-(nitrosooxy)-propan-2-ol, 2-(nitrosooxy)-propan-1-ol and 1,2-bis(nitrosooxy)propane with Tert-Butyl Nitrite

Tert-butyl nitrite (2 mL, 15.1 mmol) was added to a round bottom flask with 1,2-propanediol (11 mL, 150.3 mmol) and the obtained solution was stirred at ambient temperature. 1 mL of the reaction solution was then mixed with 7.5 mL 1,2-propanediol.

### Synthesis Example 4—Stability of Non-Aqueous Mixtures of 1-(nitrosooxy)-propan-2-ol, 2-(nitrosom)-propan-1-ol and 1,2-propanediol

Three different concentrations of 1-(nitrosooxy)-propan-2-ol and 2-(nitrosooxy)-propan-1-ol in 1,2-propanediol were prepared and stored in both a refrigerator (5° C.) and freezer (−20° C.). Aliquots of each solution were taken periodically and analysed by GC to determine the concentration of 1-(nitrosooxy)-propan-2-ol and 2-(nitrosooxy)-propan-1-ol.

The results of the GC analysis are shown in the table below (column: Rxi-5Sil MS, 20 m×0.18 mm, 0.36 film thickness; carrier: He; Inlet: 250° C., split ratio 100:1; constant flow: 1.0 mL/min; oven temperature profile: 40° C. (3 min), 10° C./min, 80° C. (0 min), 30° C./min, 250° C. (3 min); FID: 300° C., H₂ flow 30 mL/min, air flow 400 mL/min, make-up flow (N₂) 25 mL/min; internal standard: 1,1,1,3,5,5,5-heptamethyl trisiloxane):

| | Refrigerator (5° C.) | | | Freezer (−20° C.) | | |
|---|---|---|---|---|---|---|
| | Concentration (% w/w) | | | Concentration (% w/w) | | |
| Stability Sample | 1-Nitrite | 2-Nitrite | Total | 1-Nitrite | 2-Nitrite | Total |
| Start High conc. | 3.75 | 2.94 | 6.69 | 3.75 | 2.94 | 6.69 |
| Start Medium | 0.81 | 0.61 | 1.42 | 0.81 | 0.61 | 1.42 |
| conc. | | | | | | |
| Start Low conc. | 0.08 | 0.06 | 0.14 | 0.08 | 0.06 | 0.14 |
| 14 days High | 3.72 | 2.91 | 6.63 | 3.76 | 2.89 | 6.65 |
| conc. | | | | | | |
| 10 days Medium | 0.86 | 0.67 | 1.53 | 0.81 | 0.63 | 1.44 |
| conc. | | | | | | |
| 10 days Low conc. | 0.08 | 0.06 | 0.14 | 0.08 | 0.06 | 0.14 |
| 28 days High | 3.67 | 2.90 | 6.57 | 3.72 | 2.93 | 6.65 |
| conc. | | | | | | |
| 27 days Medium | 0.81 | 0.63 | 1.44 | 0.74 | 0.57 | 1.31 |
| conc. | | | | | | |
| 27 days Low conc. | 0.09 | 0.07 | 0.16 | 0.07 | 0.06 | 0.13 |
| 56 days High | 3.47 | 2.69 | 6.16 | 3.55 | 2.74 | 6.29 |
| conc. | | | | | | |
| 64 days Medium | 0.73 | 0.57 | 1.30 | 0.74 | 0.58 | 1.32 |
| conc. | | | | | | |
| 64 days Low conc. | 0.07 | 0.06 | 0.13 | 0.07 | 0.06 | 0.13 |
| 84 days High | 3.33 | 2.59 | 5.92 | 3.50 | 2.71 | 6.21 |
| conc. | | | | | | |
| 84 days Medium | 0.77 | 0.60 | 1.37 | 0.78 | 0.62 | 1.40 |
| conc. | | | | | | |
| 84 days Low conc. | 0.07 | 0.06 | 0.13 | 0.08 | 0.06 | 0.14 |

Note:
no build-up of pressure was observed for any of the samples.

### Synthesis Example 5—Solvent Free Preparation of 1-(nitrosooxy)-propan-2-ol, 2-(nitrosom)-propan-1-ol, and 1,2-bis(nitrosooxy)propane with Sodium Nitrite

Water (30 mL) and sodium nitrite (19.01 g, 272.8 mmol) were added to a 100 mL three-necked round bottom flask, flushed with nitrogen and cooled down to 1° C. on a water bath cooled with an external cooler. 1,2-Propanediol (10 mL, 136.7 mmol) was added. Concentrated sulphuric acid (7 mL, 127.4 mmol) and water (20 mL) were pre-cooled to room temperature and added dropwise during one hour via a dropping funnel. During the addition, the water layer formed a thick slurry and a green second layer was formed. Before completion of acid addition (5 mL remaining) the flask was removed from the cooling bath and the green layer was decanted into a separation funnel and washed with 2× saturated aqueous NaHCO₃ solution. The green layer faded to yellow and after separation was dried over Na₂SO₄ and filtered through a syringe filter (Acrodisc® 13 mm, 0.45 μM SUPOR®) to yield 1.1 g mixture of approximately 0.25/0.1/1 of 1-(nitrosooxy)-propan-2-ol/2-(nitrosooxy)-propan-1-ol/1,2-bis(nitrosooxy)propane. No starting-material 1,2-propanediol could be detected within the limits of NMR sensitivity.

¹H-NMR, δ ppm: 5.81-5.76 (m, br, 1.0H), 5.63 (br, 0.1H), 4.93 (br, 2.08H), 4.73-4.65 (br, m, 0.47H), 4.14 (br, 0.19H), 3.84-3.77 (br, m, 0.22H), 1.49-1.48 (br, m, 3.21H), 1.43 (br, 0.51H), 1.28 (br, 0.72H).

### Synthesis Example 6—Preparation of (2S)-1-(nitrosooxy)-propan-2-ol, (2S)-2-(nitrosom)-propan-1-ol and (2S)-1,2-bis(nitrosooxy)propane

(S)-1,2-propanediol (5 mL, 66.97 mmol), water (15 mL), dichloromethane (30 mL) and sodium nitrite (9.34 g, 134 mmol) were added to a 100 mL three-necked round bottom flask, flushed with nitrogen and cooled down to 1° C. on a water bath cooled with an external cooler. Concentrated sulphuric acid (3.5 mL, 63.69 mmol) and water (10 mL) were pre-cooled to room temperature and added dropwise via a syringe-pump during 1 h. After addition the mixture was stirred for additional 60 minutes. After separation of the two layers, the DCM layer was diluted with additional DCM (15 mL) and washed with sat. aq. NaHCO₃ (15 mL), followed by brine (15 mL), then dried over Na₂SO₄, filtered over a sintered glass filter and reduced in vacuo. The residue was taken up again in 30 mL DCM, washed with 1.4% w/w aq. bicarbonate solution, then dried over Na₂SO₄, filtered over a sintered glass filter and reduced in vacuo to yield 1 g of product mixture. The mixture of consisted of (2S)-1,2-propanediol (3%), (2S)-1-(nitrosooxy)-propan-2-ol (23%), (2S)-2-(nitrosooxy)-propan-1-ol (14%) and (2S)-1,2-bis(nitrosooxy)propane (60%) based on NMR.

¹H-NMR, δ ppm: 5.83-5.74 (m, 1.0H), 5.66-5.57 (br, 0.22H), 4.99-4.85 (br, 1.98H), 4.76-4.59 (br, 0.77H), 4.17-4.07 (br, 0.38H), 3.86-3.73 (br, 0.40H), 1.8-1.6 (br, 0.97H), 1.48 (d, J=6.7 Hz, 3.12H), 1.40 (d, J=6.6 Hz, 0.63H), 1.28 (d, J=6.5 Hz, 1.15H).

### Synthesis Example 7—Preparation of (2R)-1-(nitrosooxy)-propan-2-ol, (2R)-2-(nitrosom)-propan-1-ol and (2R)-1,2-bis(nitrosooxy)propane

(R)-1,2-propanediol (5 mL, 66.97 mmol), water (15 mL), dichloromethane (30 mL) and sodium nitrite (9.34 g, 134 mmol) were added to a 100 mL three-necked round bottom flask, flushed with nitrogen and cooled down to 1° C. on a water bath cooled with an external cooler. Concentrated sulphuric acid (3.5 mL, 63.69 mmol) and water (10 mL) were pre-cooled to room temperature and added dropwise via a syringe-pump during 1 h. After addition the mixture was stirred for additional 55 minutes. After separation of the two layers, the DCM layer was diluted with additional DCM (10 mL) and washed with saturated aqueous NaHCO₃ (20 mL), then dried over Na₂SO₄, filtered over a sintered glass filter and reduced in vacuo. The mixture of consisted of (2R)-1,2-propanediol (17%), (2R) (nitrosooxy)-propan-2-ol (16%), (2R)-2-(nitrosooxy)-propan-1-ol (7%) and (2R)-1,2-bis(nitrosooxy)propane (59%) based on NMR.

¹H-NMR, δ ppm: 5.83-5.74 (m, 1.0H), 5.66-5.57 (br, 0.12H), 4.99-4.85 (br, 2.10H), 4.76-4.59 (br, 0.53H), 4.17-4.07 (br, 0.24H), 3.86-3.73 (br, 0.28H), 2.4-2.1 (br, 0.38H), 1.48 (d, J=6.8 Hz, 3.20H), 1.40 (br, 0.56H), 1.28 (br(d), 0.88H).

### Synthesis Example 8—Preparation of 1-(nitrosooxy)propan-3-ol and 1,3-bis(nitrosooxy)propane

1,3-propanediol (2.5 g, 32.86 mmol), water (7 mL), dichloromethane (15 mL) and sodium nitrite (4.53 g, 65.7 mmol) were added to a 100 mL round bottom flask, flushed with nitrogen and cooled down to 0° C. for 15 min on a water bath cooled with an external cooler. Concentrated sulphuric acid (1.7 mL, 31.2 mmol) and water (5 mL) were pre-cooled to room temperature and added dropwise for 5 minutes. After addition the mixture was stirred for additional 60 minutes at 0° C. The two layers was then separated, and the organic phase was diluted with additional DCM (10 mL), washed with saturated aqueous NaHCO₃ (2×25 mL), dried over MgSO₄, filtered over a sintered glass filter. Finally, 1,3-propanediol (16.4 g 216 mmol) was added to the organic phase followed by removal of DCM in vacuo. Based on NMR the mixture (18.1 g) contained 1,3-propandiol (86.9 wt. %), 1-(nitrosooxy)-propan-3-ol (11.8 wt. %), and 1,3-bis(nitrosooxy)propane (1.3 wt. %).

1H-NMR, δ 4.76-4.88 (m, 2H), 3.83 (t, J=5.7 Hz, 2H), 3.73 (t, J=6.1 Hz, 2H), 2.79 (s, 1H), 2.18 (quintet, J=6.3 Hz, 2H), 1.99 (quintet, J=6.2 Hz, 2H), 1.80 (quintet, J=5.7 Hz, 2H).

### Synthesis Example 9—Scaled Up Process for the Preparation of 1-(nitrosooxy)-propan-2-ol, 2-(nitrosom)-propan-1-ol and 1,2-bis(nitrosooxy)propane with Sodium Nitrite

### 9.1 Chemicals Used

Starting materials were purchased from the list of suppliers in the table below. Unless otherwise noted the chemicals were used as received without further purification.

| List of used chemicals | | |
|---|---|---|
| and solvents | | |
| Chemical/Solvent | Grade | Supplier |
| 1,2-Propanediol | EMPROVE ® ESSENTIAL | Merck |
| | Ph. Eur. or BP or USP, ≥99% | |
| Sodium nitrite | Conforms to current ACS, | VWR, Acros |
| | USP or Ph. Eur., ≥97% | |
| Sulfuric acid | ≥95.0, Conforms to current | VWR, Acros |
| | ACS, USP or Ph. Eur. | |
| TBME | Conforms to current ACS, | VWR, Acros |
| | USP or Ph. Eur., ≥99% | |
| Sodium bicarbonate | Conforms to current ACS, | VWR, Acros |
| | USP or Ph. Eur. | |
| Magnesium sulfate | USP, dried | VWR, Acros |
| Argon | 4.8 or higher | Linde AG, |
| | | Westfalen AG |

### 9.2 General Procedure for the Synthesis of PDNO Using DCM as Solvent (Origin Process)

A round bottom flask was equipped with a stirrer and dropping funnel. Water (3.0 veq.) was added and sodium nitrite (2.0 equiv.) was charged to the flask. The solution was cooled (0° C.) and PD (1.0 equiv.) and DCM (6 rel. vol.) were also added. During further cooling, a sulfuric acid solution (1.0 eq. H₂SO₄, 2.0 rel. vol. water) was prepared. The sulfuric acid solution was further added dropwise to the reaction mixture while keeping the reaction mixture between 0° C. and 5° C. After complete addition of the acid, the solution was further stirred for 1 h to complete reaction.

Then, the reaction was quenched with saturated NaHCO₃ solution (6.0 rel. vol.). The phases were separated, and the organic layer was further washed with NaHCO₃ solution (6.0 rel. vol.). The organic phase was dried over MgSO₄, filtered, diluted with PD, and concentrated under reduced pressure using a rotary evaporator (water bath temperature 40° C.).

The product was obtained as a slightly yellowish liquid.

### 9.3 General Synthesis of PDNO Using TBME as Solvent

A round bottom flask was equipped with stirrer and dropping funnel. Argon was flushed through for several minutes. A diluted sulfuric acid solution (1.0 eq. H₂SO₄, 2.0 rel. vol. water) was prepared in advanced and precooled (−30° C.). Water was added to the flask (3.0 rel. vol.). Sodium nitrite (2.0 equiv.) was added into the water. TBME (7.5 rel. vol.) was added. Propanediol (1.0 equiv.) was added and the reaction mixture was cooled (−20° C.) flushing constantly with argon. The reaction mixture was stirred well while adding dropwise the precooled sulfuric acid. The reaction temperature was monitored during the entire addition of the acid. After addition, the reaction mixture was further stirred (30-60 min) at cold temperature (−20° C.). Afterwards, the reaction mixture was allowed to warm up (−5° C.). The reaction was stopped by quenching with saturated NaHCO₃ solution (6.0 rel. vol.). The phases were separated. The organic layer was further washed with saturated NaHCO₃ solution until a pH value of 7-8 was obtained. The organic phase was then dried over MgSO4. The crude PDNO solution (being the product comprising a mixture of 1-(nitrosooxy)-propan-2-ol, 2-(nitrosooxy)-propan-1-ol and 1,2-bis(nitrosooxy)propane) was diluted with PD (3 rel. vol.) (PD being the propanediol starting material) and further concentrated under reduced pressure at ambient temperature (25° C.).

The crude PDNO solution was further purified using a vertical tube evaporation apparatus.

PDNO was obtained as a slightly yellowish liquid.

### 9.4 Detailed Synthesis of PDNO Using TBME as Solvent

The process was designed to produce approx. 7.5 L of 7% PDNO solution with one synthesis (one “run”). The synthesis was performed several times, to give the desired batch size. GC analysis was used each single run for purity determination. The runs which are within the specifications for the organic related compounds can be blended together to yield one batch. The entire crude PDNO batch was then purified. After purification, the strong PDNO solution was then further diluted with PD to yield the desired concentration (usually 7% PDNO solution).

A suitable double wall reactor (60 L) was equipped with specific “cup-stirrer”, dropping funnel and attachment for argon. The reactor was flushed for 5 min to 10 min with a constant argon stream. Water (3.0 L) was added to the reactor. Sodium nitrite (2.0 equiv., 1886 g) was added through the reactor. The reaction was further stirred until all of the salt was dissolved. 1,2-propanediol (1.0 equiv., 1040 g, 1 L) was added, followed by tert-butylmethyl ether (7.5 rel. vol., 7.5 L). The reaction mixture was then cooled by continuous stirring and argon flow at an inner reaction temperature of −20° C. Meanwhile sulfuric acid (1.0 equiv., 1340 g, 728 mL) was diluted with water (2.0 L) and cooled at −30° C. After reaching an inner reaction temperature of −20° C., the diluted acid was added dropwise to the reaction mixture while vigorous stirring.

The stirring speed was varied during the addition of the acid. Starting with approx. 350 rpm to a slower stirring speed by the end of the reaction (approx. 180 rpm.). This variation of the stirring speed is due the two-phase reaction system and the slowly precipitation of sodium sulfate by further progress of the reaction (due to the addition of more and more sulfuric acid).

During the entire addition of the sulfuric acid, the reaction temperature was monitored. The temperature should ideally be in range of (−20±3) ° C. In addition, the reaction was stirred for 30-60 min at (−20±3) ° C.

The reaction was allowed to warm up to −5° C. to 0° C. The reaction was stopped by the addition of saturated NaHCO₃ solution (6.0 rel. vol 6.0 L) followed by the addition of water (10 L). The phases were separated and the organic layer was transferred into a separate double wall reactor and chilled at 0° C. to −5° C. The organic layer was washed several times (approx. 2-3 times) with saturated NaHCO₃ solution (4.0 rel. vol., 4.0 L). The pH value of the water phase was monitored after each washing step. The pH value was about 7-8. The water phases were discarded. The organic layer was dried over MgSO4 and filtered over a Whatman filter paper.

The crude PDNO (solution in TBME) was diluted by the addition of further PD (3.0 rel. vol., 3.0 L). This crude PDNO was transferred to a rotary evaporator and concentrated under reduced pressure. The water bath temperature during the evaporation was maintained at a maximum temperature of 25° C. The evaporation of the main amount of TBME was removed in a time range between 1.5 h and 2.0 h.

The evaporation of the organic solvents could then be continued at a water bath temperature at (0±2) ° C. for several hours using a high vacuum pump (during the development the PDNO purity was monitored at these conditions, and over a period of 6 h the product purity was not affected).

### 9.5 Further Purification of the Crude PDNO Solution

The final purification of the PDNO solution was done by vertical tube evaporation. The PDNO solution was distilled under high vacuum with a continuous thin steam of PDNO at 0° C. The storing tank for the “crude” PDNO solution was chilled at 0° C. The entire distillation was performed at 0° C. The storage tank for the “purified” PDNO was also chilled at −10° C. to 0° C. After each run of the evaporation of the entire batch PDNO, the residual organic solvent (TBME) can be checked via GC. This evaporation was continued until the desired limit for the residual solvents was achieved. In the case of PDNO the limit for the residual solvent is 1000 ppm.

### 9.6 Preparation of the Final Dilution

After purification, PDNO was further diluted to reach the favoured concentration. The first step was to filter the PDNO solution into a clean glass bottle via Whatman filter. In addition, the assay of the PDNO solution was determined via q-NMR. The amount of PD for dilution can be calculated. The PD was filtered first over a Whatman filter. The final dilution can be done at ambient temperatures. The calculated amount of PD was added to the PDNO solution (or the other way around). The resulting mixture was shaken for several minutes to obtain a homogeneous solution. The final PDNO solution was filled into the product bottles.

PDNO (7.5 kg; 7% solution) was yielded as a slightly yellowish liquid.

### Example 10—In Vitro Studies on the Antimicrobial Properties of PDNO

The antimicrobial properties of PDNO (i.e. the product that comprises one of, or a mixture of, 1-(nitrosooxy)-propan-2-ol, 2-(nitrosooxy)-propan-1-ol and 1,2-bis(nitrosooxy)propane prepared as outlined above) was tested on the bacteria B. spizizenii, S. aureus and P. aeruginosa as well as the fungi C. albicans and A. brasiliensis.

The tests were performed in solutions containing 100 ml PDNO and 600 ml NaCl-peptone having a pH in the range of 6 to 8. 100 μl diluted microorganism suspensions (10-100 cfu) were added. The solutions were filtered through a PALL Microfunnels® (GN-6 Metricel membrane 0.45 μm) and washed three times with 100 ml NaCl-peptone solution.

The filters were transferred to agar plates, TSA/CASO for B. subtilis, P. aeruginosa and S. aureus and Sabouraud glucose agar for C. albicans and A. brasiliensis, and incubated at 35° C. for at least three days for the bacteria and 25° C. for at least five days for the yeast. Table 1 show the spike and recovery of each bacteria/yeast.

TABLE 1

| Strain | Spike [cfu] | Recovery [cfu] | Recovery [%] |
|---|---|---|---|
| S. aureus | 47 | 1 | 2.1 |
| B. subtilis | 47 | 1 | 2.1 |
| P. aeruginosa | 89 | 0 | 0 |
| A. brasiliensis | 73 | 0 | 0 |
| C. albicans | 51 | 0 | 0 |

The results show that PDNO has potent antimicrobial properties as the recovery rate of all microorganisms is <2.1%.

### Example 11—In Vitro Studies on the Antiviral Properties of PDNO

The antiviral properties of PDNO were tested on the virus SARS-CoV-2.

The antiviral effect of PDNO at 40 μM, 10 μM and 2.5 μM was assessed using a yield reduction assay as follows. Confluent Vero E6 cells (Green monkey kidney cell-line) seeded in a 24-wells plate were infected with SARS-CoV-2 for one hour at a MOI of 0.005 (one infective viral particle for every 200 cells).

After one hour the viral inoculum was removed, the cells were washed with phosphate-buffered saline (PBS) and treated with 40 μM, 10 μM and 2.5 μM of PDNO in propanediol in cell culture medium wherein the combined concentration of PDNO and PD in the medium was 0.5 vol-%.

A virus control, with infected and untreated cells, and a cell control, with uninfected cells, was also created. The effect of PDNO on uninfected cells was tested by treating uninfected cells with 40 μM, 10 μM respective 2.5 μM of PDNO in PD in cell culture medium wherein the combined concentration of PDNO and PD in the medium was 0.5 vol-%.

The cell media containing the different concentrations of PDNO was collected and re-treated every two hours for 24 h with 40 μM, 10 μM respective 2.5 μM of PDNO in PD in cell culture medium wherein the combined concentration of PDNO and PD in the medium was 0.5 vol-%.

The collected cell media was used for viral RNA quantification by quantitative reverse transcription PCR (RT-qPCR).

Cell media was also collected and used for viral RNA quantification by RT-qPCR 48 h and 72 h after the start of the experiment to evaluate the lasting antiviral effect of PDNO after treatment was interrupted.

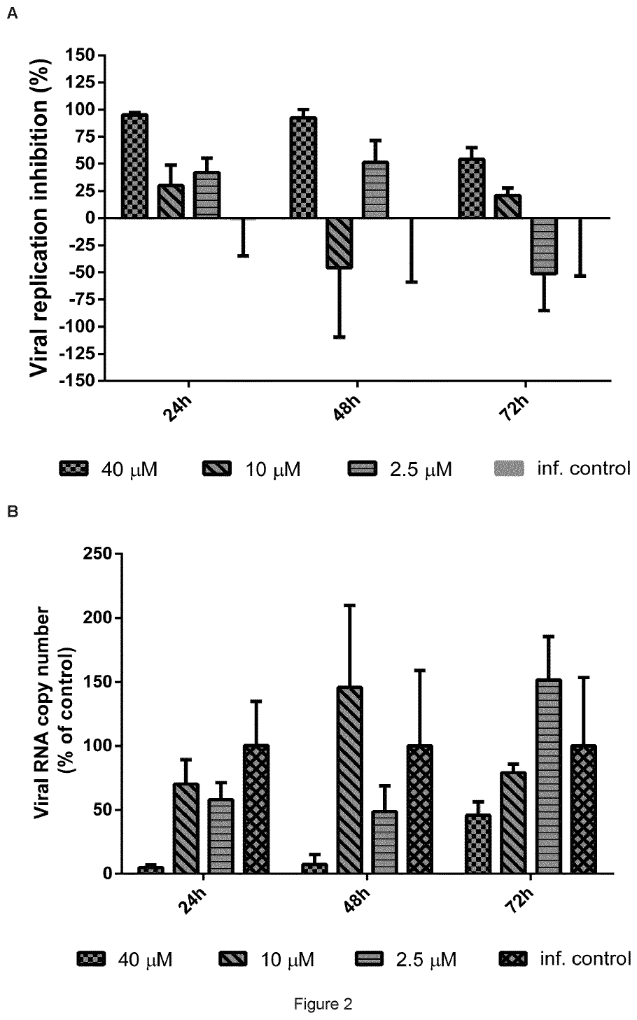

The number of viral RNA in infected cells treated with nothing, PD, 2.5 μM, 10 μM and 40 μM PDNO in PD in in cell culture medium wherein the combined concentration of PDNO and PD in the medium was 0.5 vol-% at time points 12, 18, 24 and 48 h was determined by RT-qPCR are seen in FIGS. 2A and 2B.

After 72 h, the experiment was terminated performing an MTT assay to evaluate if treatment with PDNO reduced or prevented cytopathic effect (CPE) development in cells infected with SARS-CoV-2.

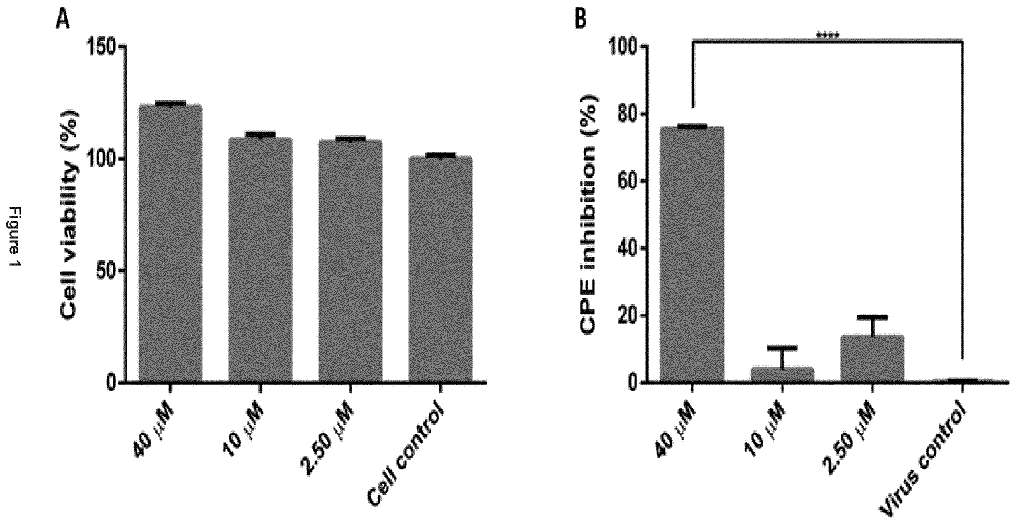

The MTT assay measures cell viability trough the quantification of formazan crystal formed by metabolically active cells. From the MTT assay results it can be observed that PDNO did not have any toxic effect at the tested concentrations while a PDNO-dependent increase in cell viability independent from the number of cells present in each well was observed in the uninfected cells (FIG. 1A), suggesting that PDNO might increase cell metabolism connected to the formation of formazan crystals.

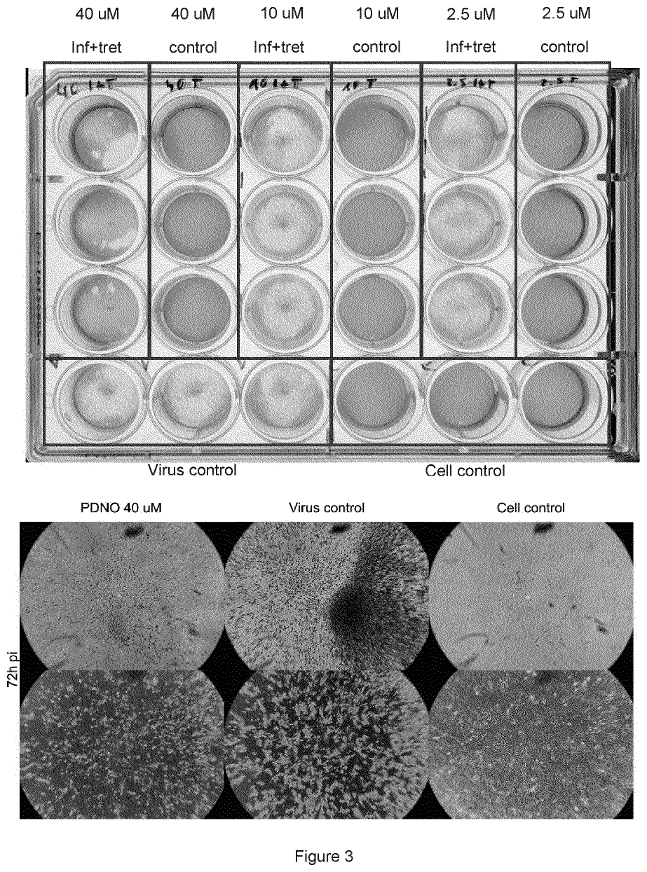

Treatment of cells with 40 μM of PDNO in PD in cell culture medium wherein the combined concentration of PDNO and PD in the medium was 0.5 vol-% resulted in a 75% reduction in CPE development in infected cells compared to untreated cells (FIG. 1B) 72 h after infection. Treatment with 10 μM or 2.5 μM of PDNO in PD in cell culture medium wherein the combined concentration of PDNO and PD in the medium was 0.5 vol-% were not effective in reducing CPE development. The antiviral effect of PDNO in PD in cell culture medium wherein the combined concentration of PDNO and PD in the medium was 0.5 vol-% solution at a PDNO concentration of 40 μM can be directly observed in FIG. 3.

As seen in FIG. 3, infected cells treated with 40 μM of PDNO in PD in cell culture medium wherein the combined concentration of PDNO and PD in the medium was 0.5 vol-% present an almost intact monolayer, except for some damage in the first two wells cause by cell drying occurred during the collection of cell supernatant. Wells treated with 10 or 2.5 μM of PDNO in PD in cell culture medium wherein the combined concentration of PDNO and PD in the medium was 0.5 vol-% present similar cell damage as observed in the infected/untreated control.

### Example 12—Determination of Plasmodium falciparum Sensitivity to PDNO

It was investigated whether PDNO shows any inhibiting potential against blood stage P. falciparum.

The multidrug resistant Dd2 Plasmodium Falciparum parasite line was maintained in culture according to standard procedures. In brief, asexual stage parasites were grown in human erythrocytes and with RPMI 1640 medium (USBiological) supplemented with 0.025 mg/mL Gentamycin (Gibco Life Technologies), 5% human serum and 5% Albumax (Gibco Life Technologies) at a hematocrit of 4%. Cultures were kept at a constant microaerophilic composition (1% O₂, 5% CO₂ and 94% N₂) and in suspension using an orbital shaker (50 rev/min) at 37° C. Cultures were prior to being drug assayed regularly stage synchronized by treatments with 5% sorbitol.

The determination of inhibitory concentrations (IC₅₀ and IC₉₀) was performed using a 9 point 2-fold dilution series of PDNO with the highest concentration set to 100 μM. The dilutions were made in PD to ensure that the same amount of PD was present for all PDNO concentrations (including the untreated control) and the total amount of PD was 0.1% of the total culture volume. The PDNO composition (or PD alone) was added once to parasites when they were at schizont stage (36+/−4 h post invasion) in triplicate wells per drug dilution. Parasites were thereafter grown for 24 h before labeled with SYBR green and MitoTracker Deep Red and assayed by flow cytometry. Parasitemia was determined using Flow Cytometry (BD FACSVerse) and data analyzed using FlowJo™ (Version 10.6.2). IC₅₀ and IC₉₀ values were computed from dose-response curves achieved by three-parametric non-linear regression using GraphPad PRISM (Version 8.4.2).

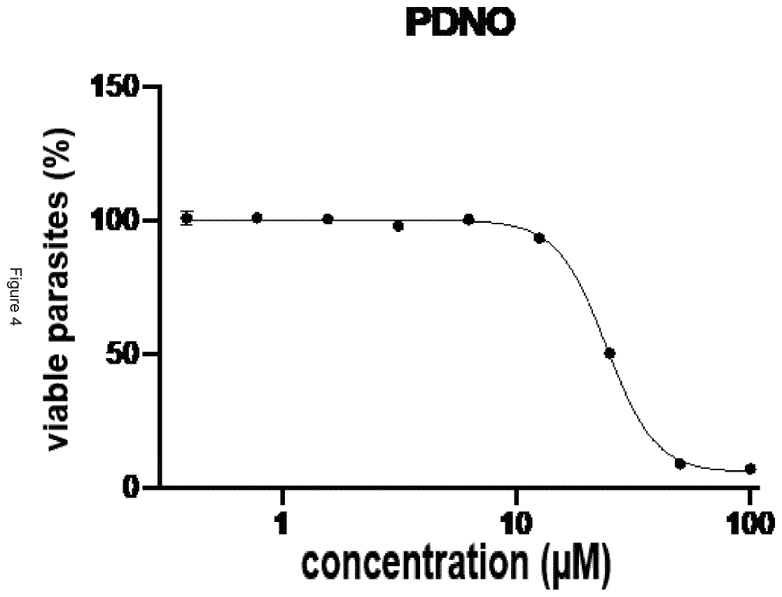

As shown in FIG. 4, the one-time administration of PDNO to the Dd2 parasite line at schizont stage revealed that the parasites are sensitive to PDNO. Resulting inhibitory concentrations on parasite viability was determined as IC50=24.2 μM and IC90=41.2 μM.

It was concluded that PDNO is effective against schizont stage P. falciparum. That the results come from NO and not PD was made sure by keeping the PD amounts constant at all PDNO concentrations used in culture.

### Example 13—Determination of Respiratory Syncytial Virus-A-Long Virus Sensitivity to PDNO

3D human bronchial epithelial MucilAir™ tissues were sourced from Epithelix Sari (Geneva, Swiss). The tissues were maintained as per the manufacturer's instruction until the day of infection and treatment. On day 0, tissues were infected apically with Respiratory Syncytial Virus-A-Long (RSV-A-Long virus) for 1.5 hours (h). The virus was aspirated and immediately thereafter the tissues were exposed basolateral to the compound PDNO (200 μM) and the solvent control PD. Infected, unexposed tissues were included as viral controls. Compound treatment was repeated at day 2, 4, and 7. Daily, the tissues were washed and the apical wash fluids (250 μl) were collected for RT-qPCR analysis to determine viral load.

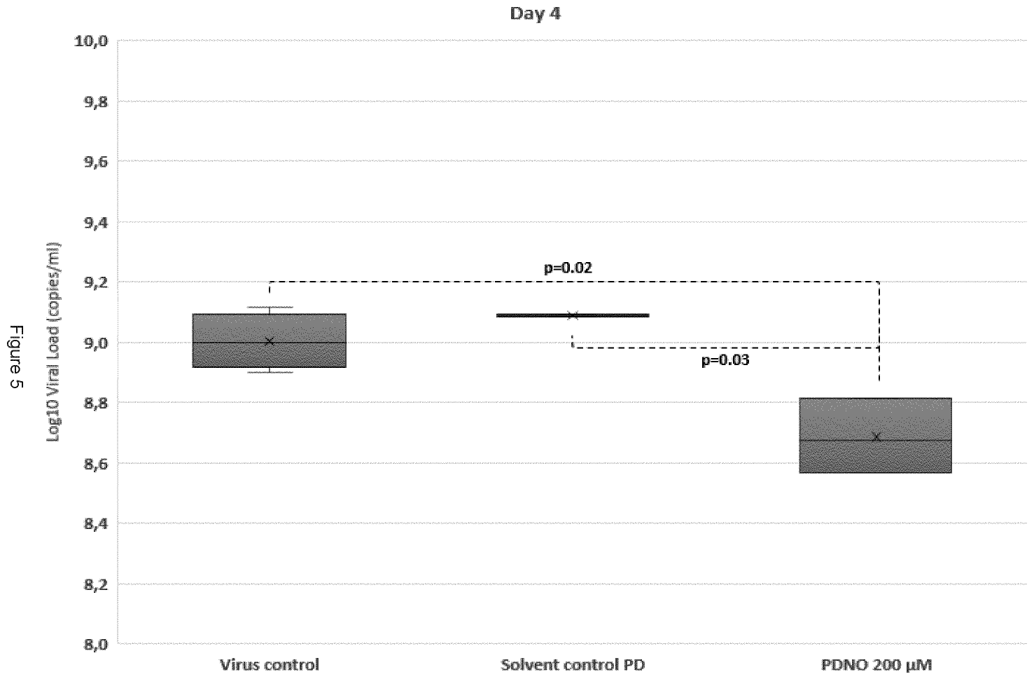

As shown in FIG. 5, the RSV log 10 viral loads (copies/ml) in MucilAir tissues at day 4 after infection and basolateral compartment treatment at day 0 and 2 with PD (solvent control) and 200 μM PDNO or the virus controls (no compound treatment). The results of one independent experiment is displayed and presented as box and whisker plots of 2 (solvent control), 3 (200 μM PDNO), and 4 (virus control) technical replicates. The p values were determined by a Student's t-Test (unpaired, unequal variance).

Conclusion: At day 4 of the experimental protocol, a significant inhibition of RSV viral load was observed for tissues treated with 200 μM PDNO compared to virus control- and solvent control (PD)-treated tissues.

1. A compound of formula (I): 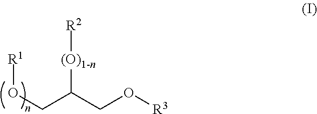
wherein R¹, R² and R³ each independently represent H or —NO,
wherein n is 0 or 1;
wherein when n is 0, R¹ is H; and
wherein when n is 1, R² is H,
provided that at least one of R¹ R² and R³ represents —NO,
for use in the treatment of a microbial infection.

2. A method of treatment of a microbial infection in a subject, which method comprises administering a compound of formula (I): 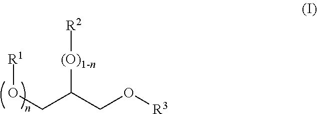
wherein R¹, R² and R³ each independently represent H or —NO,
wherein n is 0 or 1;
wherein when n is 0, R¹ is H; and
wherein when n is 1, R² is H,
provided that at least one of R¹ R² and R³ represents —NO,
to a subject in need of such treatment.

3. The use of a compound of formula (I): 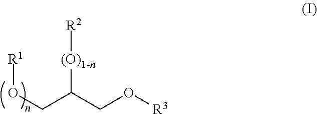
wherein R¹, R² and R³ each independently represent H or —NO,
wherein n is 0 or 1;
wherein when n is 0, R¹ is H; and
wherein when n is 1, R² is H,
provided that at least one of R¹ R² and R³ represents —NO,
for the manufacture of a medicament for a method of treatment of a microbial infection.

4. A compound for use as claimed in claim 1, a method of treatment as claimed in claim 2, or a use as claimed in claim 3, wherein the microbial infection is a parasitic, bacterial, fungal or viral infection.

5. A compound for use, a method, or a use as claimed in claim 4, wherein the bacteria is selected from the group of gram-positive bacteria.

6. A compound for use, a method, or a use as claimed in claim 5, wherein the bacteria is selected from the phylum Firmicutes, preferably wherein the bacteria is selected from the class Bacilli, preferably wherein the bacteria is selected from the order Bacillales, preferably wherein the bacteria is selected from the family Bacillaceae and/or Staphylococcaceae, preferably wherein the bacteria is selected from the genus Bacillus and/or Staphylococcus, preferably wherein the bacteria is Bacillus spizizenii and/or Staphylococcus aureus.

7. A compound for use, a method, or a use as claimed in claim 4, wherein the bacteria is selected from the group of gram-negative bacteria.

8. A compound for use, a method, or a use as claimed in claim 7, wherein the bacteria is selected from the phylum Proteobacteria, preferably wherein the bacteria is selected from the class Gammaproteobacteria, preferably wherein the bacteria is selected from the order Pseudomonadales, preferably wherein the bacteria is selected from the family Pseudomonadaceae, preferably wherein the bacteria is selected from the genus Pseudomonas, preferably wherein the bacteria is Pseudomonas aeruginosa.

9. A compound for use, a method, or a use as claimed in claim 4, wherein the fungus is selected from the division Ascomycota.

10. A compound for use, a method, or a use as claimed in claim 9 wherein the fungus is selected from the class Saccharomycetes, or wherein the fungus is selected from the order Saccharomycetales, or wherein the fungus is selected from the family Saccharomycetaceae, or wherein the fungus is selected from the genus Candida, or wherein the fungus is Candida albicans.

11. A compound for use, a method, or a use as claimed in claim 9 wherein the fungus is selected from the class Eurotiomycetes, or wherein the fungus is selected from the order Eurotiales, or wherein the fungus is selected from the family Trichocomaceae, or wherein the fungus is selected from the genus Aspergillus, or wherein the fungus is Aspergillus brasiliensis.

12. A compound for use, a method, or a use as claimed in claim 4, wherein the virus is selected from the realm Riboviria.

13. A compound for use, a method, or a use as claimed in claim 12 wherein the virus is selected from the phylum Incertae Sedis, or wherein the virus is selected from the order Nidovirales, or wherein the virus is selected from the family Coronaviridae, or wherein the virus is selected from the genus Betacoronavirus, or wherein the virus is selected from the subgenus Sarbecovirus, or wherein the virus is a SARS-CoV-2 virus.

14. A compound for use, a method, or a use as claimed in claim 12 wherein the virus is selected from the phylum Negarnaviricota, for example wherein the virus is selected from the order Monoegavirales, more specifically wherein the virus is selected from the family Paramyxoviridae, such as wherein the virus is selected from the genus Orthopneumovirus, such as wherein the infection is caused by a respiratory syncytial virus (RSV).

15. A compound for use, a method, or a use as claimed in claim 4, wherein the parasite causing the parasitic infection is a unicellular protozoan parasite.

16. A compound for use, a method, or a use as claimed in claim 15 wherein the parasite is from the genus Plasmodium, such as wherein the parasite is Plasmodium Falciparum.

17. A compound for use, a method, or a use as claimed in any of one of the preceding claims, wherein the treatment includes treatment of an infection in a patient caused by the microbe, such as the treatment includes treatment of an infection in a patient caused by the bacteria, parasite, fungus or virus.

18. A compound for use, a method, or a use as claimed in claim 17, wherein the symptoms of the infected patient include one of the symptoms selected from the list consisting of fever, aching muscles, headache, cough, fatigues, nausea, pain, tiredness, vomiting, diarrhoea, nasal congestion, a sore throat, shortness of breath, increased mucus production, weight loss and a rash.

19. A compound for use, a method, or a use as claimed in claims 17 and 18, wherein the treatment reduces or inhibits replication of the microbe, such as the parasite, bacteria, fungus and/or virus, in a patient, prevents infection of a patient by the microbe, such as the parasite, bacteria, fungus and/or virus, kills the microbe, such as the parasite, bacteria, fungus and/or virus in a patient and/or affects the immune system of a patient positively.
